(19) United States
(12) Patent Application Publication
(10) Pub. No.: US 2008/0287460 A1
(43) Pub. Date: Nov. 20, 2008

(54) NEW COMPOUNDS 835

(75) Inventors: Jeremy Nicholas Burrows, Sodertalje (SE); Sven Hellberg, Sodertalje (SE); Katharina Hogdin, Sodertalje (SE); Sofia Karlstrom, Sodertalje (SE); Karin Kolmodin, Sodertalje (SE); Johan Lindstrom, Sodertalje (SE); Can Slivo, Sodertalje (SE)

Correspondence Address: PEPPER HAMILTON LLP, 400 BERWYN PARK, 899 CASSATT ROAD, BERWYN, PA, 19312-1183, US

(73) Assignees: Astex Therapeutics Limited, Cambridge (GB); AstraZeneca AB, Sodertalje (SE)

(21) Appl. No.: 12/120,608

(22) Filed: May 14, 2008

Related U.S. Application Data
(60) Provisional application No. 60/917,999, filed on May 15, 2007.

(51) Int. Cl. A61K 31/497; C07D 233/90; A61K 31/4168; C07D 403/10; A61K 31/506; C07D 401/10; A61K 31/4439

(52) U.S. Cl. 514/255.05; 548/321.5; 514/401; 544/333; 514/256; 546/274.4; 514/341

(57) ABSTRACT

This invention relates to novel compounds having the structural formula I below:

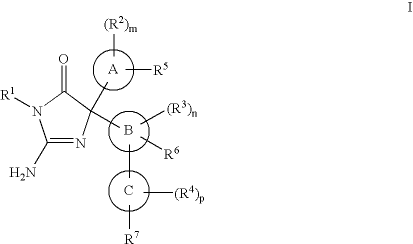

and to their pharmaceutically acceptable salt, compositions and methods of use. These novel compounds provide a treatment or prophylaxis of cognitive impairment, Alzheimer Disease, neurodegeneration and dementia.

The present invention relates to novel compounds, their pharmaceutical compositions. In addition, the present invention relates to therapeutic methods for the treatment and/or prevention of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689. WO99/64587, WO99/31236, EP1037977, WO00/17369, WO01/23533, WO0047618, WO00/58479, WO00/69262, WO01/00663, WO01/00665, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type I protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class I transmembrane protein called APP, or amyloid precursor protein. Aβ-secretase activity cleaves this protein between residues Met671 and Asp672 (numbering of 770aa isoform of APP) to form the N-terminus of Aβ. A second cleavage of the peptide is associated with γ-secretase to form the C-terminus of the Aβ peptide.

Alzheimer's disease (AD) is estimated to afflict more than 20 million people worldwide and is believed to be the most common form of dementia. Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much greater chance of developing AD, and also of developing it at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to acquire Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of APPβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of AD or fragments thereof (Yankner, 1996; De Strooper and Konig, 1999). BACE is therefore an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264, WO05/058311, WO05/097767, WO06/041404, WO06/041405, WO06/0065204, WO06/0065277, US2006287294, WO06/138265, US20050282826, US20050282825, US20060281729, WO06/138217, WO06/138230, WO06/138264, WO06/138265, WO06/138266, WO06/099379, WO06/076284, US20070004786, US20070004730, WO07/011,833, WO07/011,810, US20070099875, US20070099898, WO07/049,532).

The compounds of the present invention show beneficial properties compared to the potential inhibitors known in the art, e.g. improved potency and/or hERG selectivity.

### DISCLOSURE OF THE INVENTION

Provided herein are novel compounds that are active BACE inhibitors. Thus, in one aspect of the invention, there is provided compounds of structural formula I:

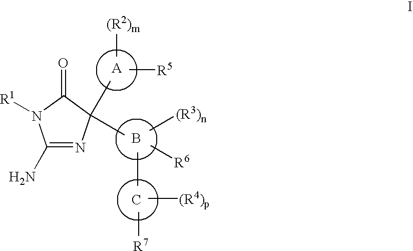

wherein
A is independently selected from hydrogen, C₁₋₆alkyl, C₃₋₆alkenyl, C₃₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl and C₀₋₆alkylheterocyclyl, wherein said C₁₋₆alkyl, C₃₋₆alkenyl, C₃₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl or C₀₋₆alkylheterocyclyl is optionally substituted with one or more R⁵;
B is independently selected from aryl and heteroaryl, said aryl or heteroaryl optionally being substituted with one or more R⁶;
C is independently selected from hydrogen, cycloalkyl, cycloalkenyl, cycloalkynyl, aryl, heteroaryl and heterocyclyl, wherein said cycloalkyl, cycloalkenyl, cycloalkynyl, aryl, heteroaryl or heterocyclyl is optionally substituted with one or more R⁷;
R¹ is selected from hydrogen, C₁₋₆alkyl, C₃₋₆alkenyl, C₃₋₆alkynyl, C₃₋₆cycloalkyl, cycloalkenyl, cycloalkynyl, aryl, heteroaryl, heterocyclyl and C₁₋₆alkylcycloalkyl, wherein said C₁₋₆alkyl, C₃₋₆alkenyl, C₃₋₆alkynyl, C₃₋₆cycloalkyl, C₅₋₇cycloalkenyl, C₅₋₇cycloalkynyl, aryl, heteroaryl or heterocyclyl, is optionally substituted with one or more D;
R², R³ and R⁴ is independently selected from N═(SO)R⁸R⁹, SF₅, and OSF₅;
R⁵, R⁶ and R⁷ is independently selected from hydrogen, halogen, nitro, CHO, C₀₋₆alkylCN, OC₁₋₆alkylCN, C₀₋₆alkylOR¹⁰, OC₂₋₆alkylOR¹¹, C₀₋₆alkylNR¹⁰R¹¹, OC₂₋₆alkylNR¹⁰R¹¹, OC₂₋₆alkylOC₂₋₆alkylNR¹⁰R¹¹, NR¹⁰OR¹¹, C₀₋₆alkylCO₂R¹⁰, OC₁₋₆alkylCO₂R¹⁰, C₀₋₆alkylCONR¹⁰R¹¹, OC₁₋₆alkylCONR¹⁰R¹¹, OC₂₋₆alkylNR¹⁰(CO)R¹¹, C₀₋₆alkylNR¹⁰(CO)R¹¹, O(CO)NR¹⁰R¹¹, NR¹⁰(CO)OR¹¹, NR¹⁰(CO)NR¹⁰R¹¹, O(CO)OR¹⁰, O(CO)R¹⁰, C₀₋₆alkylCOR¹⁰, OC₁₋₆alkylCOR¹⁰, NR¹⁰(CO)(CO)R¹¹, NR¹⁰(CO)(CO)NR¹⁰R¹¹, C₀₋₆alkylSR¹⁰, C₀₋₆alkyl(SO₂)NR¹⁰R¹¹, OC₁₋₆alkylNR¹⁰(SO₂)R¹¹, OC₀₋₆alkyl(SO₂)NR¹⁰R¹¹, C₀₋₆alkyl(SO)NR¹⁰R¹¹, OC₁₋₆alkyl(SO)NR¹⁰R¹¹, OSO₂R¹⁰, SO₃R¹⁰, C₀₋₆alkylNR¹⁰(SO₂)NR¹⁰R¹¹, C₀₋₆alkylNR¹⁰(SO)R¹¹, OC₂₋₆alkylNR¹⁰(SO)R¹¹, OC₁₋₆alkylSO₂R¹⁰, C₁₋₆alkylSO₂R¹⁰, C₀₋₆alkylSOR¹⁰, C₁₋₆alkyl, C₂₋₆alkenyl, C₂₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl and C₀₋₆alkylheterocyclyl, wherein said C₁₋₆alkyl, C₂₋₆alkenyl, C₂₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl or C₀₋₆alkylheterocyclyl is optionally substituted by one or more D;
R⁸ and R⁹ is independently selected from hydrogen, C₁₋₆alkyl, C₂₋₆alkenyl, C₂₋₆alkynyl, cycloalkyl, cycloalkenyl, cycloalkynyl, aryl, heteroaryl and heterocyclyl, wherein said C₁₋₆alkyl, C₂₋₆alkenyl, C₂₋₆alkynyl, cycloalkyl, cycloalkenyl, cycloalkynyl, aryl, heteroaryl or heterocyclyl is optionally substituted by one or more D; or
R⁸ and R⁹ may together form a 3 to 7 membered heterocyclic ring containing one or more heteroatoms selected from N, O or S, wherein said heterocyclic ring is optionally substituted by one or more D;
R¹⁰ and R¹¹ is independently selected from hydrogen, halogen, C₁₋₆alkyl, C₂₋₆alkenyl, C₂₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl, C₀₋₆alkylheterocyclyl, C₀₋₆alkylOR¹² and C₀₋₆alkylNR¹²R¹³, wherein said C₁₋₆alkyl, C₂₋₆alkenyl, C₂₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl or C₀₋₆alkylheterocyclyl is optionally substituted by one or more D; or
R¹⁰ and R¹¹ may together form a 4 to 6 membered heterocyclic ring containing one or more heteroatoms selected from N, O or S, wherein said heterocyclic ring is optionally substituted by one or more D;
R¹² and R¹³ is independently selected from hydrogen, C₁₋₆alkyl, C₃₋₆alkenyl, C₃₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheterocyclyl and C₀₋₆alkylheteroaryl, wherein said C₁₋₆alkyl, C₃₋₆alkenyl, C₃₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl or C₀₋₆alkylheterocyclyl is optionally substituted by one or more D; or
R¹² and R¹³ may together form a 4 to 6 membered heterocyclic ring containing one or more heteroatoms selected from N, O or S wherein said heterocyclic ring is optionally substituted by one or more D;
D is independently selected from halogen, nitro, CN, OR¹⁴, C₁₋₆alkyl, C₂₋₆alkenyl, C₂₋₆alkynyl, C₀₋₆alkylaryl, C₀₋₆alkylheteroaryl, C₀₋₆alkylcycloalkyl, C₀₋₆alkylcycloalkenyl, Co 6alkylcycloalkynyl, C₀₋₆alkylheterocyclyl, OC₂₋₆alkylNR¹⁴R¹⁵, NR¹⁴R¹⁵, CONR¹⁴R¹⁵, NR¹⁴(CO)R¹⁵, O(CO)C₁₋₆alkyl, (CO)OC₁₋₆alkyl, COR¹⁴, (SO₂)NR¹⁴R¹⁵, NSO₂R¹⁴, SO₂R¹⁴, SOR⁴, (CO)C₁₋₆alkylNR¹⁴R¹⁵, (SO₂)C₁₋₆alkylNR¹⁴R¹⁵, OSO₂R¹⁴ and SO₃R¹⁵, wherein said C₁₋₆alkyl, C₂₋₆alkenyl, C₂₋₆alkynyl, C₀₋₆alkylaryl, C₀₋₆alkylheteroaryl, C₀₋₆alkylheterocyclyl, C₀₋₆alkylcycloalkyl C₀₋₆alkylcycloalkenyl or C₀₋₆alkylcycloalkynyl is optionally substituted with halogen, nitro, CN, C₁₋₆alkyl, OR¹⁴, OSO₂R¹⁴ or SO₃R¹⁴;
R¹⁴ and R¹⁵ is independently selected from hydrogen, halogen, C₁₋₆alkyl, C₂₋₆alkenyl, C₂₋₆alkynyl, C₃₋₆cycloalkyl, aryl, heteroaryl and heterocyclyl; or
R¹⁴ and R¹⁵ may together form a 4 to 6 membered heterocyclic ring containing one or more heteroatoms selected from N, O or S;
m=0, 1, 2 or 3;
n=0, 1, 2 or 3;
p=0, 1, 2 or 3;
wherein one of m, n or p is at least 1;
as a free base or a pharmaceutically acceptable salt, solvate or solvate of a salt thereof.

In another aspect of the invention, there is provided compounds of formula I, wherein R¹ is C₁₋₆alkyl.

In another aspect of the invention, there is provided compounds of formula I, wherein R¹ is methyl.

In another aspect of the invention, there is provided compounds of formula I, wherein A represents phenyl.

In another aspect of the invention, there is provided compounds of formula I, wherein A is C₀₋₆alkylaryl, said C₀₋₆alkylaryl being optionally substituted with one or more R⁵.

In one embodiment of this aspect, R⁵ is selected from hydrogen and C₀₋₆alkylOR¹⁰.

In another embodiment of this aspect, said C₀₋₆alkylOR¹⁰ represents methoxy.

In another aspect of the invention, there is provided compounds of formula I, wherein B is aryl, optionally substituted with one R⁶.

In another aspect of the invention, there is provided compounds of formula I, wherein B represents phenyl substituted with one fluoro.

In another aspect of the invention, there is provided compounds of formula I, wherein C is selected from aryl and heteroaryl, wherein said aryl or heteroaryl is optionally substituted with one or more R⁷.

In one embodiment of this aspect, R⁷ is selected from hydrogen, halogen, C₀₋₆alkylCN and C₀₋₆alkylOR¹⁰.

In another aspect of the invention, there is provided compounds of formula I, wherein C represents pyrimidyl.

In another aspect of the invention, there is provided compounds of formula I, wherein C represents phenyl substituted with one methoxy.

In another aspect of the invention, there is provided compounds of formula I, wherein C represents pyridyl.

In another aspect of the invention, there is provided compounds of formula I, wherein C represents pyridyl substituted with one methoxy, one cyano or one fluoro.

In another aspect of the invention, there is provided compounds of formula I, wherein

m=0 or 1;
n=0;
p=0 or 1;
wherein one of m or p is least 1.

In one embodiment of this aspect, m is 1 and R² is independently selected from N═(SO)R⁸R⁹ and SF₅.

In another embodiment of this aspect, R² represents N═(SO)R⁸R⁹, and R⁸ and R⁹ represents methyl.

In another aspect of the invention, there is provided compounds of formula I, wherein p is 1 and R⁴ is N═(SO)R⁸R⁹.

In one embodiment of this aspect, wherein R⁸ and R⁹ represents methyl.

In another aspect of the invention, there is provided compounds of formula I, wherein m is 1 and R² is SF₅.

In another aspect of the invention, there is provided compounds of formula I, wherein

A is C₀₋₆alkylaryl, optionally substituted with one R⁵;
B is aryl, optionally substituted with one or more R⁶;
C is aryl or heteroaryl, wherein said aryl or heteroaryl is optionally substituted with one R⁷;
R¹ is C₁₋₆alkyl;
R², R³ and R⁴ is independently selected from N═(SO)R⁸R⁹ and SF₅;
R⁵, R⁶ and R⁷ is independently selected from hydrogen, halogen and C₀₋₆alkylOR¹⁰; C₀₋₆alkylCN;
R⁸ and R⁹ is C₁₋₆alkyl;
R¹⁰ is C₁₋₆alkyl;
m=0 or 1;
n=0;
p=0 or 1;
wherein one of m or p is 1.

In another aspect of the invention, there is provided compounds of formula I, wherein

A is phenyl;
B is phenyl, optionally substituted with one or more R⁶;
C is aryl or heteroaryl, wherein said aryl or heteroaryl is optionally substituted with one R⁷;
R¹ is C₁₋₆alkyl;

### R² is SF₅;

R⁶ and R⁷ is independently selected from hydrogen, halogen, C₁₋₆alkyl, C₀₋₆alkylOR¹⁰; C₀₋₆alkylCN;
m=1;
n=0;
p=0; and
R¹⁰ represents methyl.

In one embodiment of this aspect, C is a heteroaryl selected from pyridine, pyrimidine, pyrazine, thiazole and pyrazole.

In another embodiment of this aspect, C is a phenyl, substituted with one, two or three R⁷, independently selected from halogen, cyano and methoxy.

In another aspect of the invention, there is provided a compound, selected from:
2-Amino-5-(4-{[dimethyl(oxido)-λ⁴-sulfanylidene]amino}phenyl)-5-(6-fluoro-3′-methoxybiphenyl-3-yl)-3-methyl-3,5-dihydro-4H-imidazol-4-one hydrochloride;
2-Amino-5-(3′-{[dimethyl(oxido)-λ⁴-sulfanylidene]amino}-5′-methoxybiphenyl-3-yl)-3-methyl-5-phenyl-3,5-dihydro-4H-imidazol-4-one hydrochloride;
2-Amino-5-(3′-{[dimethyl(oxido)-λ⁴-sulfanylidene]amino}-5′-methoxybiphenyl-3-yl)-5-(4-methoxyphenyl)-3-methyl-3,5-dihydro-4H-imidazol-4-one hydrochloride;
2-Amino-5-(4-fluoro-3-pyrimidin-5-yl-phenyl)-3-methyl-5-[4-(pentafluoro-λ⁶-sulfanyl)phenyl]-3,5-dihydro-4H-imidazol-4-one;
2-Amino-5-(4-fluoro-3-pyrimidin-5-yl-phenyl)-3-methyl-5-[3-(pentafluoro-λ⁶-sulfanyl)phenyl]-3,5-dihydro-4H-imidazol-4-one;
5-(5-{2-Amino-1-methyl-5-oxo-4-[3-(pentafluoro-λ⁶-sulfanyl)phenyl]-4,5-dihydro-1H-imidazol-4-yl}-2-fluorophenyl)nicotinonitrile;
2-Amino-5-(4-fluoro-3-pyridin-3-yl-phenyl)-3-methyl-5-[3-(pentafluoro-λ⁶-sulfanyl)phenyl]-3,5-dihydro-4H-imidazol-4-one;
2-Amino-5-[4-fluoro-3-(5-methoxypyridin-3-yl)phenyl]-3-methyl-5-[3-(pentafluoro-λ⁶-sulfanyl)phenyl]-3,5-dihydro-4H-imidazol-4-one;
2-Amino-5-[4-fluoro-3-(5-fluoropyridin-3-yl)phenyl]-3-methyl-5-[3-(pentafluoro-λ⁶-sulfanyl)phenyl]-3,5-dihydro-4H-imidazol-4-one;
5-(5-{2-Amino-1-methyl-5-oxo-4-[4-(pentafluoro-λ⁶-sulfanyl)phenyl]-4,5-dihydro-1H-imidazol-4-yl}-2-fluorophenyl)nicotinonitrile 0.25 acetate;
2-Amino-5-(4-fluoro-3-pyridin-3-ylphenyl)-3-methyl-5-[4-(pentafluoro-λ⁶-sulfanyl)phenyl]-3,5-dihydro-4H-imidazol-4-one;
2-Amino-5-[4-fluoro-3-(5-methoxypyridin-3-yl)phenyl]-3-methyl-5-[4-(pentafluoro-λ⁶-sulfanyl)phenyl]-3,5-dihydro-4H-imidazol-4-one; and
2-Amino-5-[4-fluoro-3-(5-fluoropyridin-3-yl)phenyl]-3-methyl-5-[4-(pentafluoro-λ⁶-sulfanyl)phenyl]-3,5-dihydro-4H-imidazol-4-one 0.25 acetate as a free base or a pharmaceutically acceptable salt, solvate or solvate of a salt thereof.

In another aspect of the invention, there is provided a compound, selected from:
2-Amino-5-(4-{[dimethyl(oxido)-λ⁴-sulfanylidene]amino}phenyl)-5-(6-fluoro-3′-methoxybiphenyl-3-yl)-3-methyl-3,5-dihydro-4H-imidazol-4-one hydrochloride;
2-Amino-5-(3′-{[dimethyl(oxido)-λ⁴-sulfanylidene]amino}-5′-methoxybiphenyl-3-yl)-3-methyl-5-phenyl-3,5-dihydro-4H-imidazol-4-one hydrochloride;
2-Amino-5-(3′-{[dimethyl(oxido)-λ⁴-sulfanylidene]amino}-5′-methoxybiphenyl-3-yl)-5-(4-methoxyphenyl)-3-methyl-3,5-dihydro-4H-imidazol-4-one hydrochloride;
2-Amino-5-(4-fluoro-3-pyrimidin-5-ylphenyl)-3-methyl-5-[4-(pentafluoro-λ⁶ sulfanyl)phenyl]-3,5-dihydro-4H-imidazol-4-one;
5-(5-{2-Amino-1-methyl-5-oxo-4-[4-(pentafluoro-λ⁶-sulfanyl)phenyl]-4,5-dihydro-1H-imidazol-4-yl}-2-fluorophenyl)nicotinonitrile 0.25 acetate;
2-Amino-5-(4-fluoro-3-pyridin-3-ylphenyl)-3-methyl-5-[4-(pentafluoro-λ⁶-sulfanyl)phenyl]-3,5-dihydro-4H-imidazol-4-one;
2-Amino-5-[4-fluoro-3-(5-methoxy pyridin-3-yl)phenyl]-3-methyl-5-[4-(pentafluoro-λ⁶-sulfanyl)phenyl]-3,5-dihydro-4H-imidazol-4-one;
2-Amino-5-[4-fluoro-3-(5-methoxypyridin-3-yl)phenyl]-3-methyl-5-[4-(pentafluoro-λ⁶-sulfanyl)phenyl]-3,5-dihydro-4H-imidazol-4-one (isomer 1);
2-Amino-5-[4-fluoro-3-(5-methoxypyridin-3-yl)phenyl]-3-methyl-5-[4-(pentafluoro-λ⁶-sulfanyl)phenyl]-3,5-dihydro-4H-imidazol-4-one (isomer 2);
2-Amino-5-[4-fluoro-3-(5-fluoropyridin-3-yl)phenyl]-3-methyl-5-[4-(pentafluoro-λ⁶-sulfanyl)phenyl]-3,5-dihydro-4H-imidazol-4-one 0.25 acetate;
2-amino-5-[4-fluoro-3-(2-fluoropyridin-3-yl)phenyl]-3-methyl-5-[4-(pentafluoro-λ⁶-sulfanyl)phenyl]-3,5-dihydro-4H-imidazol-4-one 0.25 acetate;
3-(5-{2-amino-1-methyl-5-oxo-4-[4-(pentafluoro-λ⁶-sulfanyl)phenyl]-4,5-dihydro-1H-imidazol-4-yl}-2-fluorophenyl)isonicotinonitrile 0.25 acetate;
2-amino-5-(4-fluoro-3-pyrazin-2-ylphenyl)-3-methyl-5-[4-(pentafluoro-λ⁶-sulfanyl)phenyl]-3,5-dihydro-4H-imidazol-4-one 0.75 acetate;
2-amino-5-[3-(2-fluoropyridin-3-yl)phenyl]-3-methyl-5-[4-(pentafluoro-λ⁶-sulfanyl)phenyl]-3,5-dihydro-4H-imidazol-4-one;
2-amino-3-methyl-5-[4-(pentafluoro-λ⁶-sulfanyl)phenyl]-5-(3-pyrimidin-5-ylphenyl)-3,5-dihydro-4H-imidazol-4-one;
2-amino-3-methyl-5-[4-(pentafluoro-λ⁶-sulfanyl)phenyl]-5-(3-pyridin-3-ylphenyl)-3,5-dihydro-4H-imidazol-4-one;
3-(3-{2-amino-1-methyl-5-oxo-4-[4-(pentafluoro-λ⁶-sulfanyl)phenyl]-4,5-dihydro-1H-imidazol-4-yl}phenyl)pyridine-4-carbonitrile;
2-amino-5-[3-(5-fluoropyridin-3-yl)phenyl]-3-methyl-5-[4-(pentafluoro-λ⁶-sulfanyl)phenyl]-3,5-dihydro-4H-imidazol-4-one;
2-amino-3-methyl-5-[4-(pentafluoro-λ⁶-sulfanyl)phenyl]-5-(3-pyrazin-2-ylphenyl)-3,5-dihydro-4H-imidazol-4-one;
2-amino-5-(2′-fluoro-3′-methoxybiphenyl-3-yl)-3-methyl-5-[4-(pentafluoro-λ⁶-sulfanyl)phenyl]-3,5-dihydro-4H-imidazol-4-one;
2-amino-5-(2′-fluoro-3′-methoxybiphenyl-3-yl)-3-methyl-5-[4-(pentafluoro-λ⁶-sulfanyl)phenyl]-3,5-dihydro-4H-imidazol-4-one (isomer 1);
2-amino-5-(2′-fluoro-3′-methoxybiphenyl-3-yl)-3-methyl-5-[4-(pentafluoro-λ⁶-sulfanyl)phenyl]-3,5-dihydro-4H-imidazol-4-one (isomer 2);
2-amino-5-(2′-fluoro-5′-methoxybiphenyl-3-yl)-3-methyl-5-[4-(pentafluoro-λ⁶-sulfanyl)phenyl]-3,5-dihydro-4H-imidazol-4-one;
2-amino-5-(2′-fluoro-5′-carbonitrile biphenyl-3-yl)-3-methyl-5-[4-(pentafluoro-λ⁶-sulfanyl)phenyl]-3,5-dihydro-4H-imidazol-4-one;
2-amino-5-(3′-methoxybiphenyl-3-yl)-3-methyl-5-[4-(pentafluoro-λ⁶-sulfanyl)phenyl]-3,5-dihydro-4H-imidazol-4-one;
3′-{2-amino-1-methyl-5-oxo-4-[4-(pentafluoro-λ⁶-sulfanyl)phenyl]-4,5-dihydro-1H-imidazol-4-yl}biphenyl-3-carbonitrile;
2-(3-{2-amino-1-methyl-5-oxo-4-[4-(pentafluoro-λ⁶-sulfanyl)phenyl]-4,5-dihydro-1H-imidazol-4-yl}phenyl)pyridine-4-carbonitrile;
2-amino-3-methyl-5-[4-(pentafluoro-λ⁶-sulfanyl)phenyl]-5-[3-(1,3-thiazol-4-yl)phenyl]-3,5-dihydro-4H-imidazol-4-one;
2-amino-3-methyl-5-[3-(1-methyl-1H-imidazol-4-yl)phenyl]-5-[4-(pentafluoro-λ⁶-sulfanyl)phenyl]-3,5-dihydro-4H-imidazol-4-one;
5-(3-{2-amino-1-methyl-5-oxo-4-[4-(pentafluoro-λ⁶-sulfanyl)phenyl]-4,5-dihydro-1H-imidazol-4-yl}phenyl)pyridine-3-carbonitrile;
2-amino-5-(3′-chloro-2′-fluorobiphenyl-3-yl)-3-methyl-5-[4-(pentafluoro-λ⁶-sulfanyl)phenyl]-3,5-dihydro-4H-imidazol-4-one;
2-amino-5-(2′,6′-difluoro-3′-methoxybiphenyl-3-yl)-3-methyl-5-[4-(pentafluoro-λ⁶-sulfanyl)phenyl]-3,5-dihydro-4H-imidazol-4-one;
2-Amino-5-(4-fluoro-3-pyrimidin-5-ylphenyl)-3-methyl-5-[3-(pentafluoro-λ⁶-sulfanyl)phenyl]-3,5-dihydro-4H-imidazol-4-one 0.25 acetate;
5-(5-{2-Amino-1-methyl-5-oxo-4-[3-(pentafluoro-λ⁶-sulfanyl)phenyl]-4,5-dihydro-1H-imidazol-4-yl}-2-fluorophenyl)nicotinonitrile 0.25 acetate;
2-Amino-5-(4-fluoro-3-pyridin-3-ylphenyl)-3-methyl-5-[3-(pentafluoro-λ⁶-sulfanyl)phenyl]-3,5-dihydro-4H-imidazo 1-4-one;
2-Amino-5-[4-fluoro-3-(5-methoxypyridin-3-yl)phenyl]-3-methyl-5-[3-(pentafluoro-λ⁶-sulfanyl)phenyl]-3,5-dihydro-4H-imidazol-4-one; and
2-Amino-5-[4-fluoro-3-(5-fluoropyridin-3-yl)phenyl]-3-methyl-5-[3-(pentafluoro-λ⁶-sulfanyl)phenyl]-3,5-dihydro-4H-imidazol-4-one
   as a free base or a pharmaceutically acceptable salt, solvate or solvate of a salt thereof.

In another aspect of the invention, there is provided a pharmaceutical formulation comprising as active ingredient a therapeutically effective amount of a compound according to formula I in association with pharmaceutically acceptable excipients, carriers or diluents.

In another aspect of the invention, there is provided a compound according to formula I for use as a medicament.

In another aspect of the invention, there is provided use of a compound according to formula I as a medicament for treating or preventing an A-related pathology.

In another aspect of the invention, there is provided use of a compound according to formula I, as a medicament for treating or preventing an Aβ-related pathology, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided use of a compound according to formula I, in the manufacture of a medicament for treating or preventing an Aβ-related pathology.

In another aspect of the invention, there is provided use of a compound according to formula I, in the manufacture of a medicament for treating or preventing an Aβ-related pathology, wherein said Aβ-related pathology is Downs syndrome, a p-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided a method of inhibiting activity of BACE comprising contacting said BACE with a compound according to formula I.

In another aspect of the invention, there is provided a method of treating or preventing an Aβ-related pathology in a mammal, comprising administering to said patient a therapeutically effective amount of a compound according to formula I.

In another aspect of the invention, there is provided a method of treating or preventing an Aβ-related pathology in a mammal, comprising administering to said patient a therapeutically effective amount of a compound according to formula I, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided a method of treating or preventing an Aβ-related pathology in a mammal, comprising administering to said patient a therapeutically effective amount of a compound according to formula I, wherein said mammal is a human.

In another aspect of the invention, there is provided a method of treating or preventing an Aβ-related pathology in a mammal, comprising administering to said patient a therapeutically effective amount of a compound according to formula I, and at least one cognitive enhancing agent, memory enhancing agent, or choline esterase inhibitor.

In another aspect of the invention, there is provided a method of treating or preventing an Aβ-related pathology in a mammal, comprising administering to said patient a therapeutically effective amount of a compound according to formula I, and at least one cognitive enhancing agent, memory enhancing agent, or choline esterase inhibitor wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided a method of treating or preventing an Aβ-related pathology in a mammal, comprising administering to said patient a therapeutically effective amount of a compound according to formula I, and at least one cognitive enhancing agent, memory enhancing agent, or choline esterase inhibitor, wherein said mammal is a human.

Some compounds of formula may have stereogenic centres and/or geometric isomeric centres (E- and Z-isomers), and it is to be understood that the invention encompasses all such optical isomers, enantiomers, diastereoisomers, atropisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

Compounds of the invention can be used as medicaments. In some embodiments, the present invention provides compounds of formula I, or pharmaceutically acceptable salts, tautomers or in vivo-hydrolysable precursors thereof, for use as medicaments. In some embodiments, the present invention provides compounds described here in for use as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In some embodiments, the present invention provides use of compounds of formula I or pharmaceutically acceptable salts, tautomers or in vivo-hydrolysable precursors thereof, in the manufacture of a medicament for the treatment or prophylaxis of Aβ-related pathologies. In some further embodiments, the Aβ-related pathologies include such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method of inhibiting activity of BACE comprising contacting the BACE with a compound of the present invention. BACE is thought to represent the major β-secretase activity, and is considered to be the rate-limiting step in the production of amyloid-β-protein (Aβ). Thus, inhibiting BACE through inhibitors such as the compounds provided herein would be useful to inhibit the deposition of Aβ and portions thereof. Because the deposition of Aβ and portions thereof is linked to diseases such Alzheimer Disease, BACE is an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, comprising administering to a mammal (including human) a therapeutically effective amount of a compound of formula I, or a pharmaceutically acceptable salt, tautomer or in vivo-hydrolysable precursor thereof.

In some embodiments, the present invention provides a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration comprising administering to a mammal (including human) a therapeutically effective amount of a compound of formula I or a pharmaceutically acceptable salt, tautomer or in vivo-hydrolysable precursors.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula I or a pharmaceutically acceptable salt, tautomer or in vivo-hydrolysable precursors and a cognitive and/or memory enhancing agent. Cognitive enhancing agents, memory enhancing agents and choline esterase inhibitors includes, but not limited to, onepezil (Aricept), galantamine (Reminyl or Razadyne), rivastigmine (Exelon), tacrine (Cognex) and memantine (Namenda, Axura or Ebixa).

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula I or a pharmaceutically acceptable salt, tautomer or in vivo-hydrolysable precursors thereof wherein constituent members are provided herein, and a choline esterase inhibitor or anti-inflammatory agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In some embodiments, the mammal or human being treated with a compound of the invention has been diagnosed with a particular disease or disorder, such as those described herein. In these cases, the mammal or human being treated is in need of such treatment. Diagnosis, however, need not be previously performed.

The present invention also includes pharmaceutical compositions which contain, as the active ingredient, one or more of the compounds of the invention herein together with at least one pharmaceutically acceptable carrier, diluent or excipent.

The definitions set forth in this application are intended to clarify terms used throughout this application. The term “herein” means the entire application.

A variety of compounds in the present invention may exist in particular geometric or stereoisomeric forms. The present invention takes into account all such compounds, including cis- and trans isomers, E- and Z-isomers, R- and S-enantiomers, diastereomers, (D)-isomers, (L)-isomers, the racemic mixtures thereof, and other mixtures thereof, as being covered within the scope of this invention, Chiral sulfoximines (R- and S-enantiomers) can also be present in the compounds described herein. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials or by synthesis using optically active reagents, auxiliaries or catalysts. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents, positions of substituents and/or variables are permissible only if such combinations result in stable compounds.

As used in this application, the term “optionally substituted,” means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example when a substituent is methyl (i.e., CH₃), then 3 hydrogens on the carbon atom can be replaced. Examples of such substituents include, but are not limited to: halogen, CN, NH₂, OH, SO, SO₂, COOH, OC₁₋₆alkyl, CH₂OH, SO₂H, C₁₋₆alkyl, OC₁₋₆alkyl, C(═O)C₁₋₆alkyl, C(═O)OC₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂, C₅₋₆aryl, OC₅₋₆aryl, C(═O)C₅₋₆aryl, C(═O)OC₅₋₆aryl, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocycyl)₂, NH(C₅₋₆heterocyclyl), N(C₅₋₆heterocyclyl)₂, NC(═O)C₅₋₆heterocyclyl, NC(═O)(C₅₋₆heterocyclyl)₂.

As used herein, “alkyl”, used alone or as a suffix or prefix, is intended to include both branched and straight chain saturated aliphatic hydrocarbon groups having from 1 to 12 carbon atoms or if a specified number of carbon atoms is provided then that specific number would be intended. For example “C₀₋₆ alkyl” denotes alkyl having 0, 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. In the case where a subscript is the integer 0 (zero) the group to which the subscript refers to indicates that the group may be absent, i.e. there is a direct bond between the groups.

As used herein, “alkenyl” used alone or as a suffix or prefix is intended to include both branched and straight-chain alkene or olefin containing aliphatic hydrocarbon groups having from 2 to 12 carbon atoms or if a specified number of carbon atoms is provided then that specific number would be intended. For example “C₂₋₆alkenyl” denotes alkenyl having 2, 3, 4, 5 or 6 carbon atoms. Examples of alkenyl include, but are not limited to, vinyl, allyl, 1-propenyl, 1-butenyl, 2-butenyl, 3-butenyl, 2-methylbut-2-enyl, 3-methylbut-1-enyl, 1-pentenyl, 3-pentenyl and 4-hexenyl.

As used herein, “alkynyl” used alone or as a suffix or prefix is intended to include both branched and straight-chain alkyne containing aliphatic hydrocarbon groups having from 2 to 12 carbon atoms or if a specified number of carbon atoms is provided then that specific number would be intended. For example “C₂₋₆alkynyl” denotes alkynyl having 2, 3, 4, 5 or 6 carbon atoms. Examples of alkynyl include, but are not limited to, ethynyl, 1-propynyl, 2-propynyl, 3-butynyl, -pentynyl, hexynyl and 1-methylpent-2-ynyl.

As used herein, “aromatic” refers to hydrocarbonyl groups having one or more unsaturated carbon ring(s) having aromatic characters, (e.g. 4n+2 delocalized electrons) and comprising up to about 14 carbon atoms. In addition “heteroaromatic” refers to groups having one or more unsaturated rings containing carbon and one or more heteroatoms such as nitrogen, oxygen or sulphur having aromatic character (e.g. 4n+2 delocalized electrons).

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, the term “cycloalkyl” is intended to include saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, “cycloalkenyl” refers to ring-containing hydrocarbyl groups having at least one carbon-carbon double bond in the ring, and having from 4 to 12 carbons atoms.

As used herein, “cycloalkynyl” refers to ring-containing hydrocarbyl groups having at least one carbon-carbon triple bond in the ring, and having from 7 to 12 carbons atoms.

As used herein, “halo” or “halogen” refers to fluoro, chloro, bromo, and iodo. “Counterion” is used to represent a small, negatively charged species such as chloride, bromide, hydroxide, acetate, sulfate, tosylate, benezensulfonate, and the like.

As used herein, the term “heterocyclyl” or “heterocyclic” or “heterocycle” refers to a saturated, unsaturated or partially saturated, monocyclic, bicyclic or tricyclic ring (unless otherwise stated) containing 3 to 20 atoms of which 1, 2, 3, 4 or 5 ring atoms are chosen from nitrogen, sulphur or oxygen, which may, unless otherwise specified, be carbon or nitrogen linked, wherein a —CH₂— group is optionally be replaced by a —C(O)—; and where unless stated to the contrary a ring nitrogen or sulphur atom is optionally oxidised to form the N-oxide or S-oxide(s) or a ring nitrogen is optionally quarternized; wherein a ring —NH is optionally substituted by acetyl, formyl, methyl or mesyl; and a ring is optionally substituted by one or more halo. It is understood that when the total number of S and O atoms in the heterocyclyl exceeds 1, then these heteroatoms are not adjacent to one another. If the said heterocyclyl group is bi- or tricyclic then at least one of the rings may optionally be a heteroaromatic or aromatic ring provided that at least one of the rings is non-heteroaromatic. If the said heterocyclyl group is monocyclic then it must not be aromatic. Examples of heterocyclyls include, but are not limited to, piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, tetrahydropyranyl, dihydro-2H-pyranyl, tetrahydrofuranyl and 2,5-dioxoimidazolidinyl.

As used herein, “heteroaryl” or “heteroaromatic” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, thiazolyl, benzothienyl, purinyl, carbazolyl, fluorenonyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl or heteroaromatic group has 1 to about 4, 1 to about 3, or 1 to 2 heteroatoms. In some embodiments, the heteroaryl or heteroaromatic group has 1 heteroatom.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric acid.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like diethyl ether, ethyl acetate, ethanol, isopropanol, or acetonitrile are used.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

Compounds of the invention further include hydrates and solvates.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

The anti-dementia treatment defined herein may be applied as a sole therapy or may involve, in addition to the compound of the invention, conventional chemotherapy. Such chemotherapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the simultaneous, sequential or separate dosing of the individual components of the treatment. Such combination products employ the compounds of this invention.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

In some embodiments, the present invention provides a compound of formula I or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension.

If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

Compounds of the present invention have been shown to inhibit beta secretase (including BACE) activity in vitro. Inhibitors of beta secretase have been shown to be useful in blocking formation or aggregation of Aβ peptide and therefore have beneficial effects in treatment of Alzheimer's Disease and other neurodegenerative diseases associated with elevated levels and/or deposition of Aβ peptide. Therefore, it is believed that the compounds of the present invention may be used for the treatment of Alzheimer disease and disease associated with dementia Hence, compounds of the present invention and their salts are expected to be active against age-related diseases such as Alzheimer, as well as other Aβ related pathologies such as Downs syndrome and p-amyloid angiopathy. It is expected that the compounds of the present invention would most likely be used as single agents but could also be used in combination with a broad range of cognition deficit enhancement agents.

### Methods of Preparation

The present invention also relates to processes for preparing the compound of formula I as to a free base or a pharmaceutically acceptable salt thereof. Throughout the following description of such processes it is understood that, where appropriate, suitable protecting groups will be added to, and subsequently removed from the various reactants and intermediates in a manner that will be readily understood by one skilled in the art of organic synthesis. Conventional procedures for using such protecting groups as well as examples of suitable protecting groups are for example described in Protective Groups in Organic Synthesis by T. W. Greene, P. G. M Wutz, 3^{rd} Edition, Wiley-Interscience, New York, 1999. It is understood that microwaves can be used for the heating of reaction mixtures.

### Preparation of Intermediates

The process, wherein R¹, R², R³, R⁴, R⁵, R⁶, R⁷, A, B and C unless otherwise specified, are as defined hereinbefore, comprises,

(i) cross coupling of a compound of formula (II), wherein Halo is a halogen such as bromine, chlorine or iodine and R¹⁶ is an optionally substituted aryl or heteroaryl, with a compound of formula (III), wherein R¹⁷ is an optionally substituted aryl or heteroaryl, to obtain a compound of formula (IV),

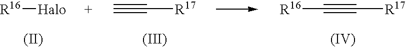

may be performed with a suitable arylhalide such as a compound of formula (I) and a suitable alkyne such as a compound of formula (III) in the presence of copper(I) iodide and a suitable palladium catalyst such as dichlorobis(benzonitrile)palladium(II), bis(triphenylphosphine)palladium(II) dichloride, palladium(II) chloride, palladium(0) tetrakistriphenylphosphine with or without a suitable ligand such as tri-tert-butylphosphine or triphenylphosphine, and a suitable base, such as trietylamine, diisopropylamine or piperidine may be used. The reaction may be performed in a solvent such as tetrahydrofuran or N,N-dimethylformamide, at temperatures between 20° C. and 100° C.
(ii) oxidative imination of a sulfoxide such as a compound of formula (VI), wherein R¹⁹ and R²⁰ are as defined for R⁸ and R⁹ above, with an appropriate amine such as a compound of formula (V), wherein R¹⁸ is an optionally substituted aryl or heteroaryl, to obtain a compound of formula (VII),

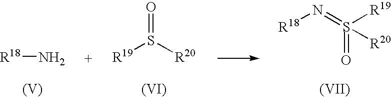

may be preformed by treating the appropriate sulfoxide with a suitable oxidation agent such as tert-butyl hypochlorite followed by addition of an appropriate amine such as a compound of formula (V) to form the azasulfoxonium chloride which upon treatment with a suitable base such as triethylamine or aqueous sodium hydroxide gives the sulfoximine. The reaction may be preformed in a solvent such as dichloromethane at temperatures between −78° C. and −30° C.
(iii) oxidation of a compound of formula (IV) to obtain a compound of formula (VIII), wherein R¹⁶ and R¹⁷ are independently chosen from an optionally substituted aryl or heteroaryl,

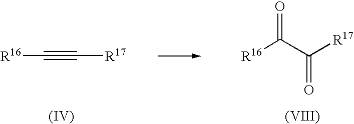

may be performed by reaction with a suitable reagent or mixture of reagents, such as sodium periodate and ruthenium dioxide, iodine and dimethyl sulfoxide, palladium chloride and dimethyl sulfoxide, oxone, hydrogen peroxide, oxygen, potassium permanganate, ruthenium(VIII) oxide, or selenium dioxide, in a suitable solvent such as dimethyl sulfoxide, dichloromethane, acetonitrile, water, acetone, chloroform or carbon tetrachloride at a temperature between −78° C. and 150° C. The reaction may be aided by the presence of a catalyst such as ruthenium(III) chloride or iron(III) chloride.
(iv) conversion of a compound of formula (VIII) to a compound of formula (IX), wherein R¹ is as defined above and R¹⁶ and R¹⁷ are independently chosen from an optionally substituted aryl or heteroaryl,

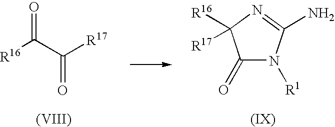

may be carried out by reaction with an appropriately N-substituted guanidine, such as N-methylguanidine, in the presence of a suitable base such as sodium carbonate or triethyl amine in a suitable solvent such as water, dioxane, ethanol or methanol, or mixtures thereof, at a temperature between 20° C. and reflux.
(v) conversion of a compound of formula VIII to obtain a compound of formula X, wherein R¹ is as defined above and R¹⁶ and R¹⁷ are independently chosen from an optionally substituted aryl or heteroaryl,

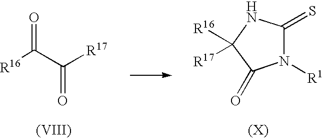

may be carried out by reaction with an appropriately N-substituted thiourea, such as N-methylthiourea, N-ethylthiourea, or N-propylthiourea, in the presence of a suitable base such as potassium hydroxide or sodium hydroxide in a suitable solvent such as water, dimethyl sulfoxide, ethanol or methanol or mixtures thereof, between 20° C. and reflux.
(vi) conversion of a compound of formula X to obtain a compound of formula IX, wherein R¹ is as defined above and R¹⁶ and R¹⁷ are independently chosen from an optionally substituted aryl or heteroaryl,

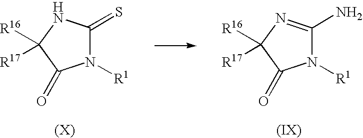

may be carried out by reaction with ammonia, or an ammonia equivalent, together with an alkylhydroperoxide such as t-butylhydroperoxide in a solvent such as ethanol, methanol or water, or a mixture thereof, at 0° C. to 50° C.
(vii) borylation of a compound of formula XI to obtain a compound of formula XII, wherein R²¹ is an optionally substituted aryl, heteroaryl or alkyne and R²² may be a group outlined in Scheme 1, wherein R²³ and R²⁴ are groups such as OH, C₁₋₆alkylO or C₂₋₃alkylO fused together to form a 5 or 6 membered boron containing heterocycle and the alkyl, cycloalkyl or aryl moieties may be optionally substituted,

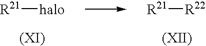

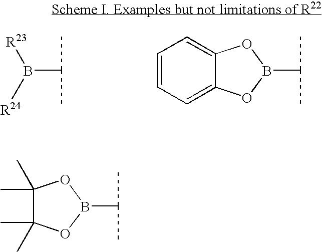

may be carried out by a reaction with:
a) an alkyllithium such as butyllithium, or magnesium, and a suitable boron compound such as trimethyl borate or triisopropyl borate. The reaction may be performed in a suitable solvent such as tetrahydrofuran, hexane or dichloromethane in a temperature range between −78° C. and 20° C.;
or,
b) a suitable boron species such as biscatecholatodiboron, bispinacolatodiboron or pinacolborane in the presence of a suitable palladium catalyst such as palladium(0) tetrakistriphenylphosphine, palladium diphenylphosphineferrocene dichloride or palladium acetate, with or without a suitable ligand such as 2-(dicyclohexylphosphino)biphenyl, and a suitable base, such as a tertiary amine, such as trietylamine or diisopropylethylamine, or potassium acetate may be used. The reaction may be performed in a solvent such as dioxane, toluene, acetonitrile, water, ethanol or 1,2-dimethoxyethane, or mixtures thereof, at temperatures between 20° C. and 160° C.
Methods of Preparation of End products

Another object of the invention is the process (a) for the preparation of compounds of general formula I, wherein R¹, R², R³, R⁴, R⁵, R⁶, R⁷, A, B and C unless otherwise specified, are defined as hereinbefore, and salts thereof. When it is desired to obtain the acid salt, the free base may be treated with an acid such as a hydrogen halide such as hydrogen chloride, sulfuric acid, a sulfonic acid such as methanesulfonic acid or a carboxylic acid such as acetic or citric acid in a suitable solvent such as tetrahydrofuran, diethyl ether, methanol, ethanol, chloroform or dichloromethane or mixtures thereof, and the reaction may occur at a temperature between −30° C. to 50° C.

(a) conversion of a compound of formula XIII to obtain a compound of formula I, wherein Halo represents a halogen such as chlorine, bromine or iodine, A, B, C, R¹, R², R³, R⁴, R⁵R⁶ and R⁷ are as defined hereinbefore,

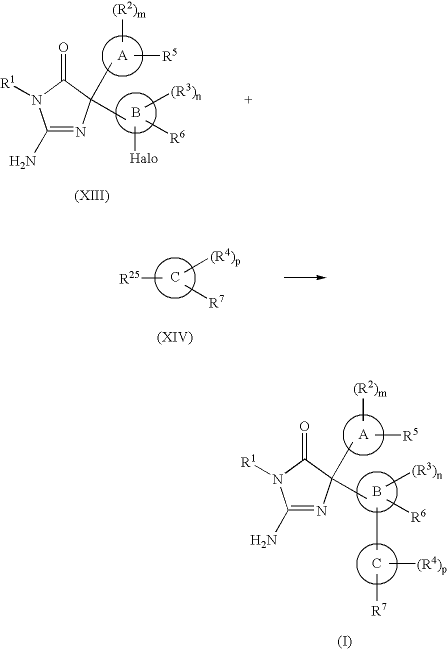

The reaction of process (a) may be carried out by a de-halogen coupling with a suitable compound of formula (XIV).

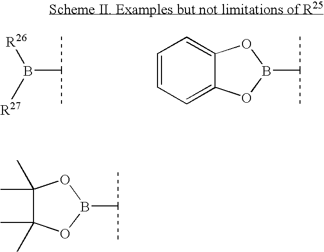

The reaction may be carried out by coupling of a compound of formula (XIII) with an appropriate aryl boronic acid or a boronic ester of formula (XIV), wherein R²⁵ may be a group outlined in Scheme II, wherein R²⁶ and R²⁷ are groups such as OH, C₁₋₆alkylO or C₂₋₃alkylO and R²⁶ and R²⁷ may be fused together to form a 5 or 6 membered boron containing heterocyclyl and the alkyl, cycloalkyl or aryl moieties may be optionally substituted. The reaction may be carried out using a suitable palladium catalyst such as tetrakis(triphenylphosphine)palladium(0), palladium diphenylphosphineferrocene dichloride, tris(dibenzylideneacetone)dipalladium(0) or palladium(II) acetate, together with or without, a suitable ligand such as 2-dicyclohexylphosphino-2′,6′-dimethoxybiphenyl (SPhos), tri-tert-butylphosphine or 2-(dicyclohexylphosphino)biphenyl, or using a nickel catalyst such as nickel on charcoal or 1,2-bis(diphenylphosphino)ethanenickel dichloride together with zinc and sodium triphenylphosphinetrimetasulfonate. A suitable base such as cesium fluoride, an alkyl amine such as triethylamine, or an alkali metal or alkaline earth metal carbonate or hydroxide such as potassium carbonate, sodium carbonate, cesium carbonate, potassium phosphate tribasic or sodium hydroxide may be used in the reaction, which may be performed in a temperature range between 20° C. and 160° C., in a suitable solvent such as toluene, tetrahydrofuran, dioxane, 1,2-dimethoxyethane, water, ethanol or N,N-dimethylformamide, or mixtures thereof.

### General Methods

Starting materials used were available from commercial sources, or prepared according to literature procedures.

Microwave heating was performed in a Creator™, Initiator™ or Smith Synthesizer™ Single-mode microwave cavity producing continuous irradiation at 2450 MHz.

Bruker av400 NMR spectrometer operating at 400 MHz ¹H equipped with a 3 mm flow injection SEI ¹H/D-¹³C probehead with Z-gradients, using a BEST 215 liquid handler for sample injection. Chemical shifts are given in ppm down- and upfield from TMS.

Resonance multiplicities are denoted s, d, t, q, m and br for singlet, doublet, triplet, quartet, multiplet, and broad respectively.

LC-MS analyses were performed on an LC-MS system consisting of a Waters Alliance 2795 HPLC, a Waters PDA 2996 diode array detector, a Sedex 75 ELS detector and a ZMD single quadrupole mass spectrometer. The mass spectrometer was equipped with an electrospray ion source (ES) operated in positive or negative ion mode. The capillary voltage was set to 3.2 kV and the cone voltage to 30 V, respectively. The mass spectrometer was scanned between m/z 100-600 by a scan time of 0.7 s. The diode array detector was scanned from 200-400 nm. The temperature of the ELS detector was adjusted to 40° C. and the pressure was set to 1.9 bar. For separation a linear gradient was applied starting at 100% A (A: 10 mM ammonium acetate in 5% acetonitrile) and ending at 100% B (B: acetonitrile). The column used was an X-Terra MS C8, 3.0 mm×50 mm, 3.5 μm (Waters) run at a flow rate of 1.0 mL/min. The column oven temperature was set to 40° C. Prep-HPLC: Preparative chromatography was run on Waters auto purification HPLC with a diode array detector. Column: XTerra MS C8, 19×300 mm, 10 μm. Varying linear gradients with acetonitrile/0.1 M ammonium acetate in 5% acetonitrile in MilliQ Water was used. Flow rate: 20 mL/min.

Thin layer chromatography (TLC) was performed on Merck TLC-plates (Silica gel 60 F₂₅₄) and spots were UV visualized. Column chromatography was performed using Merck Silica gel 60 (0.040-0.063 mm), or employing a Combi Flash® Companion™ system using RediSep™ normal-phase flash columns.

Compounds have been named using ACD/Name, version 9.0, software from Advanced Chemistry Development, Inc. (ACD/Labs), Toronto ON, Canada, www.acdlabs.com, 2004.

### EXAMPLES

Below follows a number of non-limiting examples of compounds of the invention.

### Example 1

### 4-[(3-Bromo-4-fluorophenyl)ethynyl]aniline

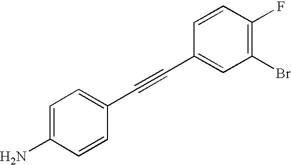

A solution of 4-ethynylaniline (820 mg, 7 mmol), 2-bromo-1-fluoro-4-iodobenzene (2.1 g, 7 mmol), copper(I) iodide (8 mg, 0.04 mmol) and bis(triphenylphosphine)palladium(II) dichloride (30 mg, 0.04 mmol) in a 2:1 mixture of tetrahydrofuran and triethylamine (18 mL) was stirred at room temperature under an atmosphere of argon overnight. The reaction mixture was concentrated in vacuo and the residue partitioned between dichloromethane (100 mL) and water (75 mL). The organic phase was separated and the aqueous phase extracted with dichloromethane. The combined organics were concentrated and purified by column chromatography, using 0-30% ethyl acetate in heptane as the eluent.

Recrystallization from diethyl ether/heptane gave 1.42 g (70% yield) of the title compound: ¹H NMR (DMSO-d₆) 7.80 (dd, J=6.8, 2.0 Hz, 1H), 7.52-7.48 (m, 1H), 7.38 (t, J=8.8 Hz, 1H), 7.22-7.18 (m, 2H), 6.58-6.54 (m, 2H), 5.60 (br s, 2H); MS (ES) m/z 290, 292 [M+H]⁺.

### Example 2

### 2-Bromo-4-[(4-{[dimethyl(oxido)-λ⁴-sulfanylidene]amino}-phenyl)ethynyl]-1-fluorobenzene

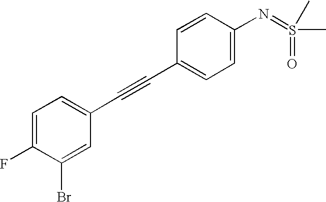

A solution of dimethyl sulfoxide (0.25 mL, 3.5 mmol) in anhydrous dichloromethane (1 mL) was added slowly to a solution of tert-butyl hypochlorite (130 mg, 1.2 mmol) in anhydrous dichloromethane at −60° C. under an atmosphere of argon. The mixture was stirred for 1 h, and then a solution of 4-[(3-bromo-4-fluorophenyl)ethynyl]aniline (290 mg, 1 mmol) in anhydrous dichloromethane (1.5 mL) was added and the resulting mixture was stirred for 4 h. A solution of triethylamine (0.25 mL) in anhydrous dichloromethane (1 mL) was added and the mixture was allowed to reach room temperature. Water was added, the organic phase was separated and the aqueous phase extracted with dichloromethane. The combined organics were concentrated and purified by column chromatography, using 20-70% ethyl acetate in heptane as the eluent, to give 110 mg (30% yield) of the title compound: ¹H NMR (DMSO-d₆): 7.87 (dd, J=6.7, 2.0 Hz, 1H), 7.58-7.54 (m, 1H), 7.41 (t, J=8.8 Hz, 1H), 7.38-7.35 (m, 2H), 6.97-6.93 (m, 2H), 3.26 (s, 6H): MS (ES) m/z 366, 368 [M−H]⁻.

### Example 3

### 1-(3-Bromo-4-fluorophenyl)-2-(4-{[dimethyl(oxido)-λ⁴-sulfanylidene]amino}phenyl)-ethane-1,2-dione

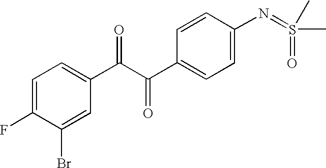

A solution of 2-bromo-4-[(4-{[dimethyl(oxido)-λ⁴-sulfanylidene]amino}phenyl)ethynyl]-1-fluorobenzene (110 mg, 0.3 mmol), palladium(II) chloride (5 mg, 0.03 mmol) in dimethyl sulfoxide (3 mL) was stirred at 140° C. for 2 h. When cooled to room temperature the mixture was diluted with water (20 mL) and extracted with dichloromethane. The combined organics were washed with brine, dried over magnesium sulfate and concentrated to give 130 mg (quantitative yield) of the title compound: MS (ES) m/z 396, 398 [M−H]⁻.

### Example 4

### 2-Amino-5-(3-bromo-4-fluorophenyl)-5-(4-{[dimethyl(oxido)-λ⁴-sulfanylidene]amino}phenyl)-3-methyl-3,5-dihydro-4H-imidazol-4-one

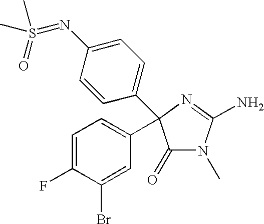

A mixture of 1-(3-bromo-4-fluorophenyl)-2-(4-{[dimethyl(oxido)-λ⁴-sulfanylidene]amino}phenyl)ethane-1,2-dione (130 mg, 0.3 mmol) and 1-methylguanidine hydrochloride (148 mg, 1.35 mmol) in dioxane (2 mL) and ethanol (2 mL) was stirred at room temperature for 20 min and a solution of sodium carbonate (143 mg, 1.35 mmol) in water (0.5 mL) was added. The resulting mixture was heated at 85° C. for 1 h, cooled to room temperature, and concentrated in vacuo. The resulting residue was partitioned between dichloromethane and water. The organic phase was separated, washed with water and brine, dried over sodium sulfate and concentrated in vacuo. Purification by column chromatography, using 5-10% 0.1 M ammonia in methanol, in dichloromethane as the eluent, gave 29 mg (21% yield) of the title compound: ¹H NMR (DMSO-d₆) 7.71 (dd, J=6.8, 2.2 Hz, 1H), 7.52-7.46 (m, 1H), 7.34 (t, J=8.8 Hz, 1H), 7.20-7.14 (m, 2H), 6.90-6.84 (m, 2H), 3.19 (s, 6 H), 3.00 (s, 3H); MS (ESI) m/z 453, 455 [M+1]⁺.

### Example 5

### 2-Amino-5-(4-{[dimethyl(oxido)-λ⁴-sulfanylidene]amino}phenyl)-5-(6-fluoro-3′-methoxybiphenyl-3-yl)-3-methyl-3,5-dihydro-4H-imidazol-4-one hydrochloride

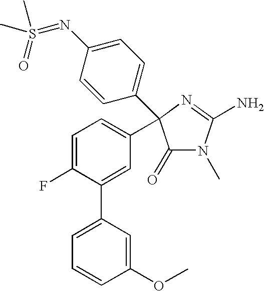

2-Amino-5-(3-bromo-4-fluorophenyl)-5-(4-{[dimethyl(oxido)-λ⁴-sulfanylidene]amino}phenyl)-3-methyl-3,5-dihydro-4H-imidazol-4-one (27 mg, 0.06 mmol), (3-methoxyphenyl)boronic acid (12 mg, 0.08 mmol), [1,1′-bis(diphenylphosphino) ferrocene]palladium(II) chloride dichloromethane adduct (3 mg, 0.003 mmol) and cesium carbonate (58 mg, 0.18 mmol) in 1,2-dimethoxyethane, water and ethanol (6:3:1, 3 mL) was irradiated in a microwave at 150° C. for 15 min. When cooled to room temperature the mixture was diluted with brine and extracted with diethyl ether. The combined organics were concentrated and purified by preparative HPLC. The acetonitrile was removed in vacuo, the residue diluted with saturated aqueous sodium hydrogen carbonate and extracted with dichloromethane. The combined organics were dried over sodium sulfate, and filtered and then hydrochloric acid (1 M in diethyl ether, 0.1 mL) was added to the filtrate. The mixture was stirred at room temperature for 5 min and the solvents were evaporated to give 14 mg (45% yield) of the title compound: ¹H NMR (DMSO-d₆) 11.56 (br s, 1H), 9.60 (br s, 2H), 7.58-7.52 (m, 1H), 7.44-7.37 (m, 3H), 7.18-7.12 (m, 2H), 7.11-7.05 (m, 2H), 7.04-6.99 (m, 1H), 6.99-6.93 (m, 2H), 3.80 (s, 3H), 3.22 (s, 6H), 3.19 (s, 3H); MS (ESI) m/z 481 [M+1]⁺.

### Example 6

### 2-Amino-5-(3-bromophenyl)-5-(4-methoxyphenyl)-3-methyl-3,5-dihydro-4H-imidazol-4-one

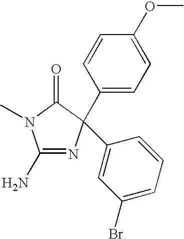

A mixture of 1-(3-bromophenyl)-2-(4-methoxyphenyl)ethane-1,2-dione (described in: Buck, J. S, and Ide, W. S. J. Am. Chem. Soc. 1930, 52, 4107-4109; 1.6 g, 4.9 mmol) and 1-methylguanidine hydrochloride (2.4 g, 22 mmol) in dioxane (50 mL) and ethanol (50 mL) was stirred at room temperature for 15 min and a solution of sodium carbonate (2.3 g, 22 mmol) in water (8 mL) was added. The resulting mixture was heated at 85° C. for 45 min, cooled to room temperature, and concentrated in vacuo. The resulting residue was partitioned between dichloromethane and water. The organic phase was separated, washed with water and brine, dried over sodium sulfate and concentrated in vacuo. Purification by column chromatography, using acetonitrile/triethylamine (95:5) as the eluent, afforded 1.6 g (94% yield) of the title compound: ¹H-NMR (DMSO-d₆) δ 7.60-7.56 (m, 1H), 7.47-7.40 (m, 2H), 7.35-7.29 (m, 2H), 7.26 (t, J=7.9 Hz, 1H), 6.89-6.83 (m, 2H), 6.68 (br s, 2H), 3.71 (s, 3H), 2.97 (s, 3H); MS (ESI) m/z 374, 376 [M+1]⁺.

### Example 7

### 5-(3-Bromo-phenyl)-3-methyl-5-phenyl-2-thioxo-imidazolidin-4-one

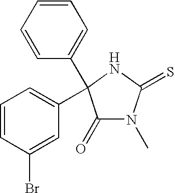

m-Bromobenzil (10.99 g, 38 mmol, described in Christy, M. E. et al. J. Med. Chem. 1977, 20, 421.) was dissolved in dimethyl sulfoxide (65 mL). N-Methylthiourea (6.85 g, 76 mmol) was added, and the solution was heated to 100° C. An aqueous solution of potassium hydroxide (1.5 M, 26 mL, 38 mmol) was added and the resulting solution was stirred at this temperature for 3 min, allowed to cool, and then poured into water (300 mL).

The resulting slurry was vigorously stirred and the pH was adjusted to below 7 with aqueous hydrochloric acid (12 M, ea 4 mL). Stirring was continued for 20 min., and the precipitate was collected by filtration. The filter cake was washed with water (150 mL) and then dried in vacuo to yield 13.98 g (100% yield) of the title compound. ¹H-NMR (DMSO-d₆): δ 11.61 (s, 1H), 7.57 (d, J=8 Hz, 1H), 7.48 (s, 1H), 7.35-7.40 (m, 5H), 7.26 (d, J=8 Hz, 2H), 3.14 (s, 3H); MS (ESI) m/z 359 and 361 [M+1]⁺.

### Example 8

### 2-Amino-5(3-bromo-phenyl)-3-methyl-5-phenyl-3, 5-4H-dihydro-imidazol-4-one

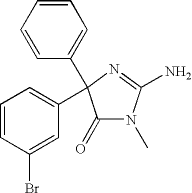

5-(3-Bromo-phenyl)-3-methyl-5-phenyl-2-thioxo-imidazolidin-4-one (2.53 g, 7 mmol) was added to a mixture of methanol (30 mL) and aqueous ammonia (25%, 10 mL). Aqueous t-butylhydroperoxide (70%, 12.5 mL, 105 mmol) was added, and the resulting mixture was stirred at 35° C. for 2 h. The mixture was then poured into water (300 ml.) and extracted with dichloromethane (3×30 mL). The combined organic phases were washed with water (200 mL), dried over magnesium sulfate and concentrated in vacuo. The residue was dissolved in dichloromethane:methanol 90:10 (20 mL), suction filtered through a silica pad and concentrated in vacuo. Recrystallization from chloroform gave 1.48 g (68% yield) of the title compound. ¹H-NMR (DMSO-d₆): δ 7.61 (s, 1H), 7.40-7.50 (m, 4H), 7.22-7.32 (m, 4H), 6.72 (s, 2H), 2.98 (s, 3H); MS (ESI) m/z 344 and 346 [M+1]⁺.

### Example 9

### 1-Bromo-3-{[dimethyl(oxido)-λ⁴-sulfanylidene]amino}-5-methoxybenzene

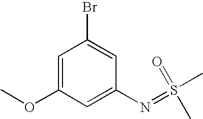

The title compound was synthesized as described for Example 2 in 8% yield, starting from 3-bromo-5-methoxyaniline (described in Hodgson, H. H. and Wignall, J. S, J. Chem. Soc., 1926, 2077-2079): ¹H NMR (DMSO-d₆) δ ppm 6.69-6.67 (m, 1H) 6.66-6.64 (m, 1H) 6.45-6.42 (m, 1H) 3.71 (s, 3H) 3.23 (s, 6H); MS (CI) m/z 278, 280 [M+1]⁺.

### Example 10

### 2-(3-{[Dimethyl(oxido)-λ⁴-sulfanylidene]amino}-5-methoxyphenyl)-4,4,5,5-tetramethyl-1,3,2-dioxaborolane

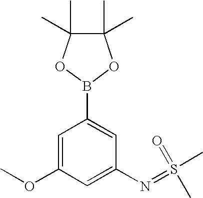

1-Bromo-3-{[dimethyl(oxido)-λ⁴-sulfanylidene]amino}-5-methoxybenzene (140 mg, 0.5 mmol), 4,4,4′,4′,5,5,5′,5′-octamethyl-2,2′-bi-1,3,2-dioxaborolane (141 mg, 0.55 mmol), [1,1′-bis(diphenylphosphino)ferrocene]palladium(II) chloride dichloromethane adduct (21 mg, 0.03 mmol), potassium acetate (74 mg, 0.75 mmol) and 1,4-dioxane (3 mL) were added to a vial and irradiated in a microwave at 150° C. for 15 min. When cooled to room temperature the mixture was filtered and the filtrate concentrated in vacuo. The resulting residue was purified on a silica gel column and eluted with 40-100% ethyl acetate in heptane to give 133 mg (81% yield) of the title compound: ¹H NMR (DMSO-d₆) δ ppm 6.90-6.87 (m, 1H) 6.73-6.70 (m, 1H) 6.57-6.53 (m, 1H) 3.70 (s, 3 f1) 3.17 (s, 6 H) 1.28 (s, 12H); MS (ES) m/z 326 [M+1]⁺.

### Example 11

### 2-Amino-5-(3′-{[dimethyl(oxido)-λ⁴-sulfanylidene]amino}-5′-methoxybiphenyl-3-yl)-3-methyl-5-phenyl-3,5-dihydro-4H-imidazol-4-one hydrochloride

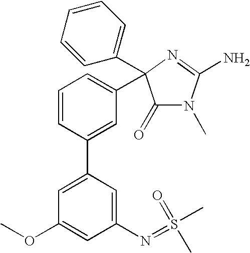

2-Amino-5-(3-bromo-phenyl)-3-methyl-5-phenyl-3,5-4H-dihydro-imidazol-4-one (69 mg, 0.2 mmol), 2-(3-{[dimethyl(oxido)-λ⁴-sulfanylidene]amino}-5-methoxyphenyl)-4,4,5,5-tetramethyl-1,3,2-dioxaborolane (65 mg, 0.2 mmol), [1,1′-bis(diphenylphosphino)ferrocene]palladium(II) chloride dichloromethane adduct (8 mg, 0.01 mmol) and potassium carbonate (83 mg, 0.6 mmol) in tetrahydrofuran (2 mL) and water (0.5 mL) were mixed and irradiated in a microwave at 130° C. for 15 min. When cooled to room temperature the mixture was diluted with brine and extracted with ethyl acetate (3×3 mL). The combined organics were concentrated in vacuo and the resulting residue was dissolved in methanol and purified by preparative HPLC. The product was dissolved in dichloromethane and methanol, then hydrochloric acid (1 M in diethyl ether, 0.5 mL) was added and the mixture was concentrated to give 57 mg (57% yield) of the title compound: ¹H NMR (DMSO-d₆) δ ppm 11.79 (br. s., 1H) 9.67 (br. s., 2H) 7.67-7.63 (m, 1H) 7.61-7.59 (m, 1H) 7.54-7.49 (m, 1H) 7.46-7.41 (m, 3H) 7.41-7.36 (m, 3H) 6.72-6.68 (m, 2H) 6.53-6.50 (m, 1H) 3.75 (s, 3H) 3.23 (s, 6H) 3.21 (s, 3H); MS (ES) m/z 463 [M+H]⁺.

### Example 12

### 2-Amino-5-(3′-{[dimethyl(oxido)-4-sulfanylidene]amino}-5′-methoxybiphenyl-3-yl)-5-(4-methoxyphenyl)-3-methyl-3,5-dihydro-4H-imidazol-4-one hydrochloride

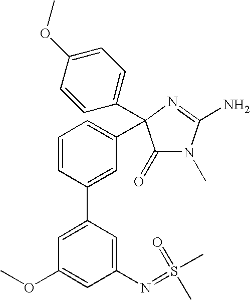

The title compound was synthesized as described for Example 11 in 64% yield, starting from 2-Amino-5-(3-bromophenyl)-5-(4-methoxyphenyl)-3-methyl-3,5-dihydro-4H-imidazol-4-one: ¹H NMR (DMSO-d₆) δ ppm 11.66 (br. s., 1H) 9.64 (br. s., 2H) 7.67-7.61 (m, 1H) 7.59-7.56 (m, 1H) 7.54-7.47 (m, 1H) 7.39-7.34 (m, 1H) 7.30-7.25 (m, 2H) 7.03-6.97 (m, 2H) 6.73-6.68 (m, 2H) 6.54-6.50 (m, 1H) 3.76 (s, 6H) 3.23 (s, 6H) 3.20 (s, 3H); MS (ES) m/z 493 [M+H]⁺.

### Example 13

### 2-Bromo-1-fluoro-4-{[4-(pentafluoro-λ⁶-sulfanyl)phenyl]ethynyl}benzene

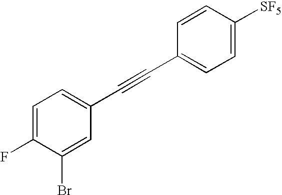

The compound was synthesized as described for Example 1 starting from 2-bromo-4-ethynyl-1-fluorobenzene (1 g, 5.02 mmol) and 4-iodophenylsulphur pentafluoride (1.658 g, 5.02 mmol) to give the title compound (1.67 g, 83% yield): ¹H NMR (400 MHz, CDCl₃) δ ppm 7.73-7.79 (m, 3H); 7.59 (d, J=8.8 Hz, 2H); 7.45-7.50 (m, 1H); 7.14 (t, J=8.5 Hz, 1H).

### Example 14

### 1-bromo-3-{[4-(pentafluoro-λ⁶-sulfanyl)phenyl]ethynyl}benzene

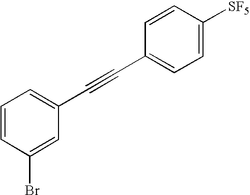

The compound was synthesized as described for Example 1 starting from 1-bromo-3-ethynylbenzene (4.11 g, 22.72 mmol) and 4-Iodophenylsulphur pentafluoride (7.5 g 22.72 mmol). Title compound was not isolated, used directly in next step Example 17. GC-MS (CI) m/z 385, 383 [M+1]⁺

### Example 15

### 2-Bromo-1-fluoro-4-{[3-(pentafluoro-λ⁶-sulfanyl)phenyl]ethynyl}benzene

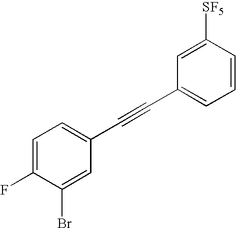

The compound was synthesized as described for Example 1 starting from 2-bromo-4-ethynyl-1-fluorobenzene (2.0 g, 10.0 mmol) and 3-iodophenylsulphur pentafluoride (3.32 g, 10.0 mmol) to give the title compound (3.24 g, 80% yield): ¹H NMR (400 MHz, CDCl₃) δ ppm 7.90-7.93 (m, 1H); 7.78 (dd, J=6.6, 2.0 Hz, 1H); 7.74 (dd, J=8.3, 1.5 Hz, 1H); 7.64 (d, J=7.6 Hz, 1H); 7.45-7.51 (m, 2H); 7.14 (t, J=8.3 Hz, 1H).

### Example 16

### 1-(3-Bromo-4-fluorophenyl)-2-[4-(pentafluoro-λ⁶-sulfanyl)phenyl]ethane-1,2-dione

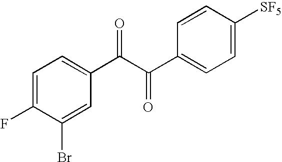

The compound was synthesized as described for Example 3 starting from 2-bromo-1-fluoro-4-{[4-(pentafluoro-λ⁶-sulfanyl)phenyl]ethynyl}benzene (1.67 g, 4.16 mmol). The product was purified on a silica column using ethyl acetate (0-50%) in n-heptane as the eluent to give the title compound (1.29 g, 71% yield): ¹H NMR (400 MHz, CDCl₃) δ ppm 8.18 (dd, J=6.6, 2.0 Hz, 1H); 8.02 (d, J=8.8 Hz, 2H); 7.86-7.90 (m, 1H); 7.85 (d, J=9.1 Hz, 2H); 7.18-7.23 (m, 1H).

### Example 17

### 1-(3-bromophenyl)-2-[4-(pentafluoro-λ⁶-sulfanyl)phenyl]ethane-1,2-dione

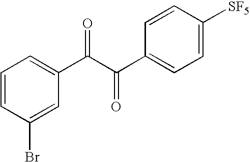

The compound was synthesized as described for Example 3 starting from 1-bromo-3-{[4-(pentafluoro-λ⁶-sulfanyl)phenyl]ethynyl}benzene (10.68 g, 27.87 mmol). The product was purified on a silica column using ethyl acetate (0-10%) in n-heptane as the eluent to give the title compound (4.64 g, 40% yield) of the title compound: GC-MS (CI) m/z 417, 415 [M+1]⁺

### Example 18

### 1-(3-Bromo-4-fluorophenyl)-2-[3-(pentafluoro-λ⁶-sulfanyl)phenyl]ethane-1,2-dione

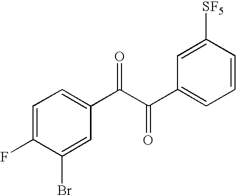

The compound was synthesized as described for Example 3 starting from 2-bromo-1-fluoro-4-{[3-(pentafluoro-λ⁶-sulfanyl)phenyl]ethynyl}benzene (3.24 g, 8.08 mmol) to give the title compound (2.82 g, 81% yield): ¹H NMR (400 MHz, CDCl₃) δ ppm 8.44 (t, J=1.9 Hz, 1H); 8.28 (dd, J=6.6, 2.3 Hz, 1H); 8.05-8.13 (m, 2H); 7.95-8.01 (m, 1H); 7.67 (t, J=8.0 Hz, 1H); 7.29 (t, J=8.1 Hz, 1H).

### Example 19

### 2-Amino-5-(3-bromo-4-fluorophenyl)-3-methyl-5-[4-(pentafluoro-λ⁶-sulfanyl)phenyl]-3,5-dihydro-4H-imidazol-4-one

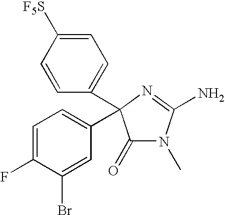

The compound was synthesized as described for Example 4 starting from 1-(3-bromo-4-fluorophenyl)-2-[4-(pentafluoro-λ⁶-sulfanyl)phenyl]ethane-1,2-dione (0.4 g, 0.92 mmol) to give the title compound (0.295 g, 65% yield): ¹H NMR (400 MHz, CDCl₃) δ ppm 7.76 (dd, J=6.6, 2.3 Hz, 1H); 7.71 (d, J=9.1 Hz, 2H); 7.63 (d, J=8.8 Hz, 2H); 7.43-7.47 (m, 1H); 7.07 (t, J=8.3 Hz, 1H); 3.13 (s, 3H); MS (ES) m/z 488,0 [M+H]⁺.

### Example 20

### 2-amino-5-(3-bromophenyl)-3-methyl-5-[4-(pentafluoro-λ⁶-sulfanyl)phenyl]-3,5-dihydro-4H-imidazol-4-one

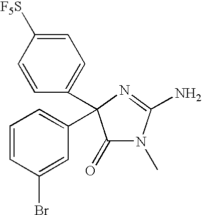

The compound was synthesized as described for Example 4 starting from 1-(3-bromophenyl)-2-[4-(pentafluoro-λ⁶-sulfanyl)phenyl]ethane-1,2-dione (4.64 g, 11.18 mmol), to give the title compound (4.44 g, 84% yield) ¹H NMR (400 MHz, MeOH) δ ppm 7.81 (d, 2H) 7.54-7.59 (m, 3H) 7.46-7.49 (m, 1H) 7.35-7.39 (m, 1H) 7.27 (t, 1H) 3.13 (s, 3H); MS (ES) m/z 472, 470 [M+1]⁺.

### Example 21

### 2-Amino-5-(3-bromo-4-fluorophenyl)-3-methyl-5-[3-(pentafluoro-λ⁶-sulfanyl)phenyl]-3,5-dihydro-4H-imidazol-4-one

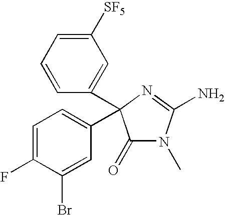

The compound was synthesized as described for Example 4 starting from 1-(3-bromo-4-fluorophenyl)-2-[3-(pentafluoro-λ⁶-sulfanyl)phenyl]ethane-1,2-dione (2.82 g, 6.51 mmol) to give the title compound (2.60 g, 82% yield): ¹H NMR (400 MHz, CDCl₃) δ ppm 7.96 (d, J=1.5 Hz, 1H); 7.64-7.77 (m, 3H); 7.38-7.48 (m, 2 H); 7.07 (t, J=8.5 Hz, 1H); 3.13 (s, 3H); MS (ES) m/z 489.9 [M+H]⁺.

### Example 22

### 2-Amino-5-(4-fluoro-3-pyrimidin-5-ylphenyl)-3-methyl-5-[4-(pentafluoro-λ⁶-sulfanyl)phenyl]-3,5-dihydro-4H-imidazol-4-one

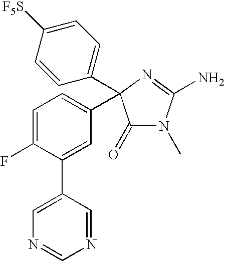

2-Amino-5-(3-bromo-4-fluorophenyl)-3-methyl-5-[4-(pentafluoro-λ⁶-sulfanyl)phenyl]-3,5-dihydro-4H-imidazol-4-one (0.295 g, 0.60 mmol), [1,1′-bis(diphenylphosphino)ferrocene]dichloropalladium(II) (complex with dichloromethane (1:1) (0.024 g, 0.03 mmol), potassium acetate (0.119 g, 1.21 mmol) and pyrimidin-5-yl boronic acid (0.074 g, 0.60 mmol) were dissolved in degassed DME/water (4:1, 4 mL). The reaction mixture was irradiated in a microwave at 120° C. for 30 minutes. The reaction mixture was filtered through celite, the filtrate was washed with ethyl acetate and concentrated. The residue was dissolved in DMSO (2 mL) and purified by preparative HPLC to give the title compound (0.150 g, 49% yield). 1H NMR (400 MHz, CDCl₃) δ ppm 9.22 (s, 1H); 8.91 (d, J=10.3 Hz, 2H); 7.70-7.74 (m, 2H); 7.59-7.69 (m, 4H); 7.20 (t, J=9.3 Hz, 1H); 3.14 (s, 3H); MS (ES) m/z 488,0 [M+H]⁺.

### Example 23

### 5-(5-{2-Amino-1-methyl-5-oxo-4-[4-(pentafluoro-A-sulfanyl)phenyl]-4,5-dihydro-1H-imidazol-4-yl}-2-fluorophenyl)nicotinonitrile 0.25 acetate

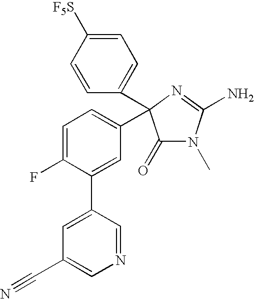

The compound was synthesized as described for Example 22 starting from 2-amino-5-(3-bromo-4-fluorophenyl)-3-methyl-5-[4-(pentafluoro-λ⁶-sulfanyl)phenyl]-3,5-dihydro-4H-imidazol-4-one (110 mg, 0.23 mmol) and 5-(4,4,5,5-tetramethyl-1,3,2-dioxaborolan-2-yl)nicotinonitrile (52 mg, 0.23 mmol) to give the title compound (49 mg, 41% yield): ¹H NMR (400 MHz, CD₃OD) δ ppm 8.89-8.97 (m, 2H); 8.37 (s, 1H); 7.79 (d, J=8.8 Hz, 2H); 7.54-7.65 (m, 4H); 7.29 (dd, J=10.1, 8.8 Hz, 1H); 3.11-3.15 (m, 3H); 2.03 (s, 0.4H); MS (ES) m/z 512.0 [M+H]⁺.

### Example 24

### 2-Amino-5-(4-fluoro-3-pyridin-3-ylphenyl)-3-methyl-5-[4-(pentafluoro-λ⁶-sulfanyl)phenyl]-3,5-dihydro-4H-imidazol-4-one

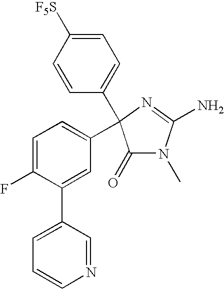

The compound was synthesized as described for Example 22 starting from 2-amino-5-(3-bromo-4-fluorophenyl)-3-methyl-5-[4-(pentafluoro-λ⁶-sulfanyl)phenyl]-3,5-dihydro-4H-imidazol-4-one (100 mg, 0.20 mmol) and pyridin-3-ylboronic acid (25 mg, 0.20 mmol) to give the title compound (55 mg, 55% yield): ¹H NMR (400 MHz, CD₃OD) δ ppm 8.67 (s, 1H); 8.55 (d, J=4.8 Hz, 1H); 7.99 (d, J=8.1 Hz, 1H); 7.80 (d, J=8.8 Hz, 2H); 7.49-7.62 (m, 5H); 7.26 (dd, J=10.0, 9.0 Hz, 1H); 3.14 (s, 3H); MS (ES) m/z 487.1 [M+H]⁺.

### Example 25

### 2-Amino-5-[4-fluoro-3-(5-methoxypyridin-3-yl)phenyl]-3-methyl-5-[4-(pentafluoro-λ⁶-sulfanyl)phenyl]-3,5-dihydro-4H-imidazol-4-one

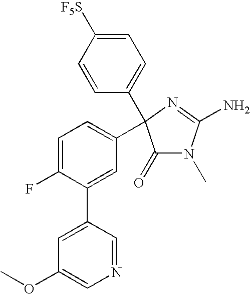

The compound was synthesized as described for Example 22 starting from 2-amino-5-(3-bromo-4-fluorophenyl)-3-methyl-5-[4-(pentafluoro-λ⁶-sulfanyl)phenyl]-3,5-dihydro-4H-imidazol-4-one (100 mg, 0.20 mmol) and 3-methoxy-5-(4,4,5,5-tetramethyl-1,3,2-dioxaborolan-2-yl)pyridine (48 mg, 0.20 mmol) to give the title compound (57 mg, 52% yield): ¹H NMR (400 MHz, CDCl₃) δ ppm 8.36 (s, 1H); 8.31 (d, J=2.8 Hz, 1H); 7.66-7.73 (m, 4 H); 7.62 (dd, J=7.2, 2.4 Hz, 1H); 7.51-7.56 (m, 1H); 7.33-7.36 (m, 1H); 7.15 (dd, J=10.1, 8.8 Hz, 1H); 3.90 (s, 3H); 3.14 (s, 3H); MS (ES) m/z 517.0 [M+H]⁺.

### Example 26

### Chromatographic preparation of the enantiomers of 2-Amino-5-[4-fluoro-3-(5-methoxypyridin-3-yl)phenyl]-3-methyl-5-[4-(pentafluoro-λ⁶-sulfanyl)phenyl]-3,5-dihydro-4H-imidazol-4-one

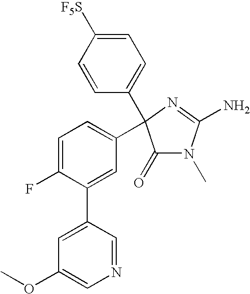

2-Amino-5-[4-fluoro-3-(5-methoxypyridin-3-yl)phenyl]-3-methyl-5-[4-(pentafluoro-λ⁶-sulfanyl)phenyl]-3,5-dihydro-4H-imidazol-4-one (0.93 g, 1.80 mmol) was dissolved in 2-propanol (30 mL) and the resulting solution was divided into five equal portions. Chiral separation was carried out on a Chiralpak IA column (50×300 mm), using 2-propanol in heptane (18:82) as eluent at a flow rate of 120 mL/min. The separation was monitored at 254 nm and the two isomers were collected and concentrated in vacuo.

Isomer 1, the first isomer to elute (0.40 g, 37% yield): ¹H NMR (400 MHz, CDCl₃) δ ppm 8.36 (s, 1H); 8.31 (d, J=2.8 Hz, 1H); 7.66-7.73 (m, 4H); 7.62 (dd, J=7.2, 2.4 Hz, 1H); 7.51-7.56 (m, 1H); 7.33-7.36 (m, 1H); 7.15 (dd, J=10.1, 8.8 Hz, 1H); 3.90 (s, 3H); 3.14 (s, 3H); MS (ES) m/z 517.0 [M+H]⁺.
Isomer 2, the second isomer to elute (0.40 g, 37% yield): ¹H NMR (400 MHz, CDCl₃) δ ppm 8.36 (s, 1H); 8.31 (d, J=2.8 Hz, 1H); 7.66-7.73 (m, 4H); 7.62 (dd, J=7.2, 2.4 Hz, 1H); 7.51-7.56 (m, 1H); 7.33-7.36 (m, 1H); 7.15 (dd, J=10.1, 8.8 Hz, 1H); 3.90 (s, 3H); 3.14 (s, 3H); MS (ES) m/z 517.0 [M+H]⁺.

### Example 27

### 2-Amino-5-[4-fluoro-3-(5-fluoropyridin-3-yl)phenyl]-3-methyl-5-[4-(pentafluoro-λ⁶-sulfanyl)phenyl]-3,5-dihydro-4H-imidazol-4-one 0.25 acetate

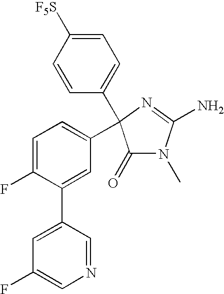

The compound was synthesized as described for Example 22 starting from 2-amino-5-(3-bromo-4-fluorophenyl)-3-methyl-5-[4-(pentafluoro-λ⁶-sulfanyl)phenyl]-3,5-dihydro-4H-imidazol-4-one (100 mg, 0.20 mmol) and 3-fluoro-5-(4,4,5,5-tetramethyl-1,3,2-dioxaborolan-2-yl)pyridine (46 mg, 0.20 mmol) to give the title compound (49 mg, 46% yield): ¹H NMR (400 MHz, CDCl₃) 8 ppm 8.60 (d, J=1.5 Hz, 1H); 8.48 (d, J=2.8 Hz, 1H); 7.70-7.74 (m, 2H); 7.56-7.69 (m, 5H); 7.18 (dd, J=10.1, 8.8 Hz, 1H); 3.14 (s, 3H) 2.11 (s, 0.6H); MS (ES) m/z 505.0 [M+H]⁺.

### Example 28

### 2-amino-5-[4-fluoro-3-(2-fluoropyridin-3-yl)phenyl]-3-methyl-5-[4-(pentafluoro-λ⁶-sulfanyl)phenyl]-3,5-dihydro-4H-imidazol-4-one 0.25 acetate

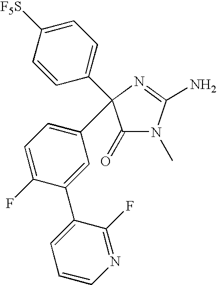

The compound was synthesized as described for 22 starting from 2-amino-5-(3-bromo-4-fluorophenyl)-3-methyl-5-[4-(pentafluoro-λ⁶-sulfanyl)phenyl]-3,5-dihydro-4H-imidazol-4-one (100 mg, 0.20 mmol) and 2-fluoropyridin-3-ylboronic acid (29 mg, 0.20 mmol) to give the title compound (18 mg, 18%): ¹H NMR (400 MHz, CDCl₃) δ ppm 8.25 (d, J=4.8 Hz, 1H); 7.80-7.86 (m, 1H); 7.69-7.74 (m, 2H); 7.62-7.67 (m, 2H); 7.52-7.58 (m, 2H); 7.26-7.31 (m, 1H); 7.16 (t, J=9.4 Hz, 4H); 5.47 (br. s., 2H); 3.14 (s, 3H); 2.08 (s, 1.1H); MS (ES) m/z 505.0 [M+H]⁺.

### Example 29

### 3-(5-{2-amino-1-methyl-5-oxo-4-[4-(pentafluoro-λ⁶-sulfanyl)phenyl]-4,5-dihydro-1H-imidazol-4-yl}-2-fluorophenyl)isonicotinonitrile 0.25 acetate

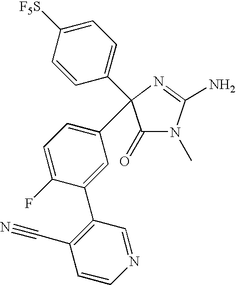

The compound was synthesized as described for 22 starting from 2-amino-5-(3-bromo-4-fluorophenyl)-3-methyl-5-[4-(pentafluoro-k-sulfanyl)phenyl]-3,5-dihydro-4H-imidazol-4-one (200 mg, 0.41 mmol) and 3-(4,4,5,5-tetramethyl-1,3,2-dioxaborolan-2-yl)isonicotinonitrile (94 mg, 0.41 mmol) to give the title compound (22 mg, 10%): ¹H NMR (400 MHz, CDCl₃) δ ppm 8.84 (s, 1H); 8.79 (d, J=5.1 Hz, 1H); 7.73 (d, J=8.8 Hz, 2H); 7.58-7.69 (m, 5H); 7.23 (t, J=9.4 Hz, 1H); 3.13 (s, 3H); 2.07 (s, 0.9H); MS (ES) m/z 512.0 [M+H]⁺.

### Example 30

### 2-amino-5-(4-fluoro-3-pyrazin-2-ylphenyl)-3-methyl-5-[4-(pentafluoro-λ⁶-sulfanyl)phenyl]-3,5-dihydro-4H-imidazol-4-one 0.75 acetate

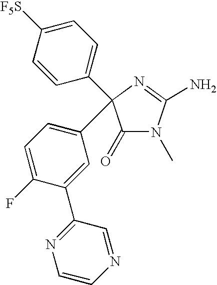

2-Bromopyrazine (163 mg, 1.02 mmol), bis(triphenylphosphine)palladium(II) chloride (18 mg, 0.03 mmol) was dissolved in DMF (2 mL) under Ar atm. The hexamethylditin (0.214 mL, 1.02 mmol) was added and the reaction mixture was heated to 130° C. for 30 minutes with MW. 2-amino-5-(3-bromo-4-fluorophenyl)-3-methyl-5-[4-(pentafluoro-λ⁶-sulfanyl)phenyl]-3,5-dihydro-4H-imidazol-4-one (250 mg, 0.51 mmol), cesium fluoride (0.076 mL, 2.05 mmol) and additional 2 mol % of bis(triphenylphosphine)palladium(II) chloride was added, and the reaction mixture was heated to 130° C. for 2 h. The reaction mixture was filtrated through a short silica column using EtOAc as eluent. The solution was concentrated in vacuo. The residue was dissolved in DMSO (2 mL) and purified by preparative HPLC to give the title compound (22 mg, 8%): ¹H NMR (400 MHz, CDCl₃) δ ppm 8.97-8.99 (m, 1H); 8.59-8.61 (m, 1H); 8.47 (d, J=2.5 Hz, 1H); 8.04 (dd, J=7.1, 2.5 Hz, 1H); 7.62-7.67 (m, 2H); 7.54-7.59 (m, 2H); 7.44-7.49 (m, 1H); 7.12 (dd, J=10.48, 8.72 Hz, 1H); 3.08 (s, 3H); 1.99 (s, 2H); MS (ES) m/z 488.0 [M+H]⁺.

### Example 31

### 2-amino-5-[3-(2-fluoropyridin-3-yl)phenyl]-3-methyl-5-[4-(pentafluoro-λ⁶-sulfanyl)phenyl]-3,5-dihydro-4H-imidazol-4-one

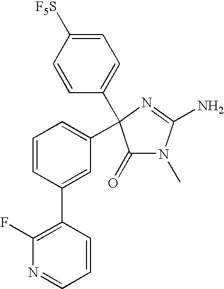

The compound was synthesized as described for Example 22 starting from 1-(3-bromophenyl)-2-[4-(pentafluoro-λ⁶-sulfanyl)phenyl]ethane-1,2-dione (100 mg, 0.21 mmol), 2-Fluoropyridyl-3-boronic acid (39.0 mg, 0.28 mmol), to give the title compound (yield 17%) of the title product: 1 ¹H NMR (400 MHz, CDCl₃) δ ppm 8.11 (d, J=4.55 Hz, 1H) 7.69-7.90 (m, 1H) 7.56-7.69 (m, 5H) 7.34-7.54 (m, 3H) 7.17-7.24 (m, 1H) 4.99 (br. s., 2H) 3.07 (s, 3H); MS (ES) m/z 487 [M+1]⁺.

### Example 32

### 2-amino-3-methyl-5-[4-(pentafluoro-λ⁶-sulfanyl)phenyl]-5-(3-pyrimidin-5-ylphenyl)-3,5-dihydro-4H-imidazol-4-one

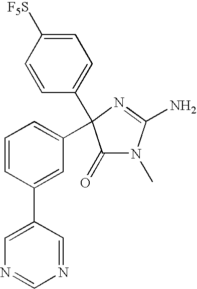

The compound was synthesized as described for example 22 starting from 1-(3-bromophenyl)-2-[4-(pentafluoro-λ⁶-sulfanyl)phenyl]ethane-1,2-dione (100 mg, 0.21 1 mmol) and pyrimidine-5-boronic acid (34.3 mg, 0.28 mmol) to give the title compound 59 mg (59% yield): ¹H NMR (400 MHz, CDCl₃) δ ppm 9.24 (s, 1H) 8.91 (s, 2H) 7.71-7.78 (m, 3H) 7.59-7.69 (m, 3 H) 7.50-7.58 (m, 2H) 6.48 (br. s., 2H) 3.20 (s, 3H); MS (ES) m/z 470 [M+1]⁺.

### Example 33

### 2-amino-3-methyl-5-[4-(pentafluoro-λ⁶-sulfanyl)phenyl]-5-(3-pyridin-3-ylphenyl)-3,5-dihydro-4H-imidazol-4-one

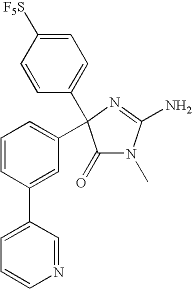

The compound was synthesized as described for example 22 starting from 1-(3-bromophenyl)-2-[4-(pentafluoro-λ⁶-sulfanyl)phenyl]ethane-1,2-dione (100 mg, 0.21 1 mmol) and Pyridine-3-boronic acid (34 mg, 0.28 mmol) to give the title compound 53 mg (53% yield): ¹H NMR (400 MHz, CD₃OD) δ ppm 8.73 (d, J=2.27 Hz, 1H) 8.51 (dd, J=4.80, 1.52 Hz, 1H) 7.97-8.11 (m, 1H) 7.75-7.86 (m, 2H) 7.66 (s, 1H) 7.58-7.64 (m, 3H) 7.45-7.54 (m, 3H) 3.14 (s, 3H); MS (ES) m/z 469 [M+1]⁺.

### Example 34

### 3-(3-{2-amino-1-methyl-5-oxo-4-[4-(pentafluoro-λ⁶-sulfanyl)phenyl]-4,5-dihydro-1H-imidazol-4-yl}phenyl)pyridine-4-carbonitrile

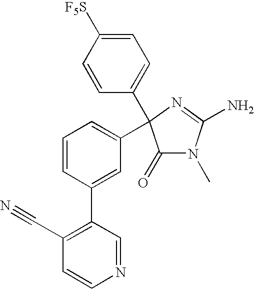

The compound was synthesized as described for example 22 starting from 1-(3-bromophenyl)-2-[4-(pcntafluoro-λ⁶-sulfanyl)phenyl]ethane-1,2-dione (100 mg, 0.21 mmol) and 3-(4,4,5,5-tetramethyl-1,3,2-dioxaborolan-2-yl)isonicotinonitrile (63.6 mg, 0.28 mmol) to give the title compound (38 mg, 36% yield): ¹H NMR (400 MHz, CD₃OD) δ ppm 8.82 (br. s., 1H) 8.74 (d, 1H) 7.80-7.83 (m, 2H) 7.77-7.80 (m, 1H) 7.67-7.71 (m, 1H) 7.64-7.66 (m, 1H) 7.62-7.64 (m, 1H) 7.58-7.61 (m, 1H) 7.54-7.58 (m, 2H) 3.15 (s, 3H); MS (ES) m/z 494 [M+1]⁺.

### Example 35

### 2-amino-5-[3-(5-fluoropyridin-3-yl)phenyl]-3-methyl-5-[4-(pentafluoro-λ⁶ sulfanyl)phenyl]-3,5-dihydro-4H-imidazol-4-one

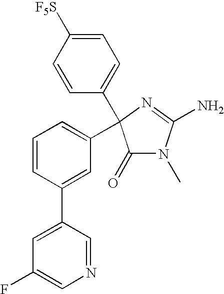

The compound was synthesized as described for example 22 starting from 1-(3-bromophenyl)-2-[4-(pentafluoro-λ⁶-sulfanyl)phenyl]ethane-1,2-dione (100 mg, 0.21 mmol) and 3-fluoro-5-(4,4,5,5-tetramethyl-1,3,2-dioxaborolan-2-yl)pyridine (61.7 mg, 0.28 mmol) to give the title compound (65 mg, 63% yield): ¹H NMR (400 MHz, CD₃OD) δ ppm 8.65 (br. s., 1H) 8.47 (d, J=2.78 Hz, 1H) 7.89 (dt, J=9.85, 2.02 Hz, 1H) 7.80-7.84 (m, 2H) 7.72 (br. s., 1H) 7.65-7.70 (m, 1H) 7.60-7.65 (m, 2H) 7.51-7.56 (m, 2H) 3.17 (s, 3H); MS (ES) m/z 487 [M+1]⁺

### Example 36

### 2-amino-3-methyl-5-[4-(pentafluoro-λ⁶-sulfanyl)phenyl]-5-(3-pyrazin-2-ylphenyl)-3,5-dihydro-4H-imidazol-4-one

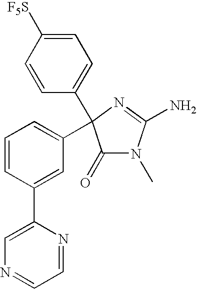

The compound was synthesized as described for example 30 starting from 1-(3-bromophenyl)-2-[4-(pentafluoro-λ⁶-sulfanyl)phenyl]ethane-1,2-dione (150 mg, 0.32 mmol) and 2-(4,4,5,5-tetramethyl-1,3,2-dioxaborolan-2-yl)pyrazine (85 mg, 0.41 mmol) to give the title compound (8.2 mg, 5.5% yield): ¹H NMR (400 MHz, CD₃OD) δ ppm 9.06 (s, 1H) 8.63-8.73 (m, 1H) 8.47-8.57 (m, 1H) 8.16 (s, 1H) 7.96-8.09 (m, 1H) 7.80 (d, J=9.09 Hz, 2H) 7.62 (d, J=8.59 Hz, 2H) 7.52 (d, J=5.31 Hz, 2H) 3.16 (s, 3H); MS (ES) m/z 470 [M+1]⁺

### Example 37

### 2-amino-5-(2′-fluoro-3′-methoxybiphenyl-3-yl)-3-methyl-5-[4-(pentafluoro-λ⁶-sulfanyl)phenyl]-3,5-dihydro-4H-imidazol-4-one

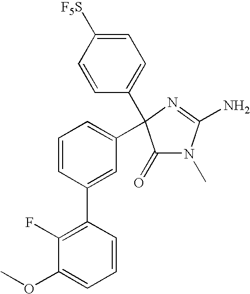

The compound was synthesized as described for example 22 starting from 1-(3-bromophenyl)-2-[4-(pentafluoro-λ⁶-sulfanyl)phenyl]ethane-1,2-dione (150 mg, 0.32 mmol) and 2-Fluoro-3-methoxyphenylboronic acid (70 mg, 0.41 mmol) to give the title compound (120 mg, 73% yield):

The title compound 2-amino-5-(2′-fluoro-3′-methoxybiphenyl-3-yl)-3-methyl-5-[4-(pentafluoro-λ⁶-sulfanyl)phenyl]-3,5-dihydro-4H-imidazol-4-one (0120 g, 0.30 mmol) was dissolved in 2-propanol (3 mL) and the resulting solution was divided into six equal portions. Chiral separation was carried out on a Berger Multigram II system using Chiralpak AD (21.2×250 mm), using 2-propanol in CO₂ (20:80) with 0.1% diethyl amine as eluent at a flow rate of 50 mL/min, after. The separation was monitored at 220 nm and the two isomers were collected and concentrated in vacuo.

Isomer 1, the first isomer to elute (47 mg, 29% yield): ¹H NMR (400 MHz, CD₃OD) 8 ppm 7.81 (dt, 2H) 7.63 (d, J=8.59 Hz, 2H) 7.54-7.59 (m, 1H) 7.37-7.50 (m, 3H) 7.06-7.17 (m, J=17.49, 8.84, 8.68, 8.68 Hz, 2H) 6.93-6.98 (m, 1H) 3.94 (s, 3H) 3.15 (s, 3H); MS (ES) m/z 516 [M+1]⁺
Isomer 2, the second isomer to elute (46 mg, 28% yield): ¹H NMR (400 MHz, CD₃OD) δ ppm 7.81 (dt, 2H) 7.63 (d, J=8.59 Hz, 2H) 7.54-7.59 (m, 1H) 7.37-7.50 (m, 3H) 7.06-7.17 (m, J=17.49, 8.84, 8.68, 8.68 Hz, 2H) 6.93-6.98 (m, 1H) 3.94 (s, 3H) 3.15 (s, 3H); MS (ES) m/z 516 [M+1]⁺

### Example 38

### 2-amino-5-(2′-fluoro-5′-methoxybiphenyl-3-yl)-3-methyl-5-[4-(pentafluoro-λ⁶ sulfanyl)phenyl]-3,5-dihydro-4H-imidazol-4-one

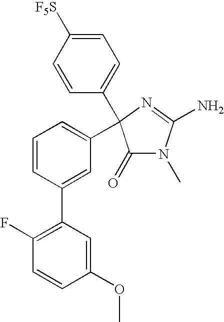

The compound was synthesized as described for example 22 starting from 1-(3-bromophenyl)-2-[4-(pentafluoro-λ⁶-sulfanyl)phenyl]ethane-1,2-dione (150 mg, 0.32 mmol) and 2-Fluoro-5-methoxyphenylboronic acid (54 mg, 0.32 mmol) to give the title compound (93 mg, 56% yield): ¹H NMR (400 MHz, CD₃OD) δ ppm 7.81 (dt, 2H) 7.61-7.67 (m, 2H) 7.54-7.58 (m, 1H) 7.38-7.52 (m, 3H) 7.11 (t, 1H) 6.88-6.95 (m, 2H) 3.81 (s, 3H) 3.16 (s, 3H); MS (ES) m/z 516 [M+1]⁺

### Example 39

### 2-amino-5-(2′-fluoro-5′-carbonitrile biphenyl-3-yl)-3-methyl-5-[4-(pentafluoro-λ⁶-sulfanyl)phenyl]-3,5-dihydro-4H-imidazol-4-one

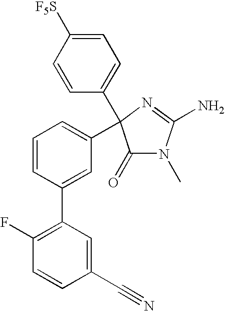

The compound was synthesized as described for example 22 starting from 1-(3-bromophenyl)-2-[4-(pentafluoro-6-sulfanyl)phenyl]ethane-1,2-dione (150 mg, 0.32 mmol) and 5-Cyano-2-fluorophenylboronic acid (63 mg, 0.38 mmol) to give the title compound (91 mg, 56% yield): ¹H NMR (400 MHz, CD₃OD) δ ppm 7.88 (dd, J=7.07, 2.27 Hz, 1H) 7.80-7.83 (m, 1H) 7.75-7.80 (m, 2H) 7.57-7.63 (m, 3H) 7.50-7.55 (m, 1H) 7.47-7.50 (m, 2H) 7.36-7.42 (m, 1H) 3.15 (s, 3H); MS (ES) m/z 511 [M+1]⁺

### Example 40

### 2-amino-5-(3′-methoxybiphenyl-3-yl)-3-methyl-5-[4-(pentafluoro-λ⁶-sulfanyl)phenyl]-3,5-dihydro-4H-imidazol-4-one

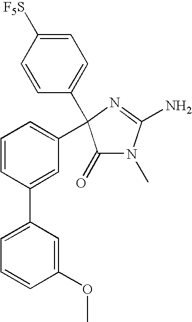

The compound was synthesized as described for example 22 starting from 1-(3-bromophenyl)-2-[4-(pentafluoro-λ⁶-sulfanyl)phenyl]ethane-1,2-dione (150 mg, 0.32 mmol) and 3-Methoxyphenylboronic acid (58 mg, 0.38 mmol) to give the title compound (83 mg, 52% yield): ¹H NMR (400 MHz, CD₃OD) δ ppm 7.79-7.90 (m, 2H) 7.61-7.66 (m, 3H) 7.55-7.60 (m, 1H) 7.43 (t, 1H) 7.32-7.39 (m, 2H) 7.08-7.15 (m, 2H) 6.88 (dd, 1H) 3.84 (s, 3H) 3.16 (s, 3H); MS (ES) m/z 499 [M+1]⁺

### Example 41

### 3′-{2-amino-1-methyl-5-oxo-4-[4-(pentafluoro-λ⁶-sulfanyl)phenyl]-4,5-dihydro-1H-imidazol-4-yl}biphenyl-3-carbonitrile

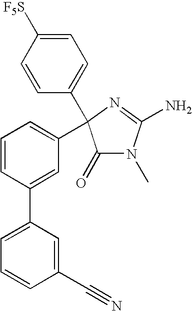

The compound was synthesized as described for example 22 starting from 1-(3-bromophenyl)-2-[4-(pentafluoro-λ⁶-sulfanyl)phenyl]ethane-1,2-dione (150 mg, 0.32 mmol) and 3-Cyanophenylboronic acid (56 mg, 0.38 mmol) to give the title compound (112 mg, 71% yield): ¹H NMR (400 MHz, CD₃OD) δ ppm 7.94 (br. s., 1H) 7.88 (d, J=8.08 Hz, 1H) 7.80 (d, J=8.84 Hz, 2H) 7.66-7.72 (m, 2H) 7.59-7.64 (m, 4H) 7.45-7.51 (m, 2H) 3.16 (s, 3H); MS (ES) m/z 493 [M+1]⁺

### Example 42

### 2-(3-{2-amino-1-methyl-5-oxo-4-[4-(pentafluoro-λ⁶-sulfanyl)phenyl]-4,5-dihydro-1H-imidazol-4-yl}phenyl)pyridine-4-carbonitrile

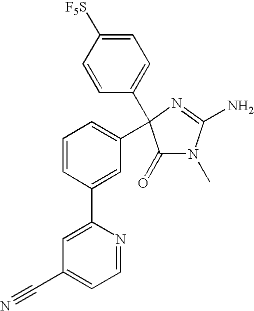

The compound was synthesized as described for example 22 starting from 1-(3-bromophenyl)-2-[4-(pentafluoro-λ⁶-sulfanyl)phenyl]ethane-1,2-dione (120 mg, 0.26 mmol) and 4-cyanopyridin-2-ylboronic acid (45 mg, 0.31 mmol) to give the title compound (3.8 mg, 3% yield): ¹H NMR (400 MHz, CD₃OD) δ ppm 8.81 (d, J=4.80 Hz, 1H) 8.17 (d, J=9.60 Hz, 2H) 7.96-8.06 (m, 1H) 7.79 (d, J=8.84 Hz, 2H) 7.57-7.68 (m, 3H) 7.48-7.56 (m, 2H) 3.13 (s, 3H); MS (ES) m/z 494 [M+1]⁺

### Example 43

### 2-amino-3-methyl-5-[4-(pentafluoro-λ⁶-sulfanyl)phenyl]-5-[3-(1,3-thiazol-4-yl)phenyl]-3,5-dihydro-4H-imidazol-4-one

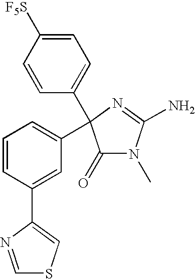

The compound was synthesized as described for example 22 starting from 1-(3-bromophenyl)-2-[4-(pentafluoro-λ⁶-sulfanyl)phenyl]ethane-1,2-dione (120 mg, 0.26 mmol) and 4-(4,4,5,5-tetramethyl-1,3,2-dioxaborolan-2-yl)thiazole (54 mg, 0.26 mmol) to give the title compound (58 mg, 48% yield): ¹H NMR (400 MHz, CD₃OD) δ ppm 9.05 (d, J=2.02 Hz, 1H) 7.96-8.04 (m, 2H) 7.88-7.95 (m, 3H) 7.66 (d, J=8.84 Hz, 2H) 7.52 (t, J=7.83 Hz, 1H) 7.33-7.37 (m, 1H) 3.29 (s, 3H); MS (ES) m/z 475 [M+1]⁺

### Example 44

### 2-amino-3-methyl-5-[3-(1-methyl-1H-imidazol-4-yl)phenyl]-5-[4-(pentafluoro-λ⁶-sulfanyl)phenyl]-3,5-dihydro-4H-imidazol-4-one

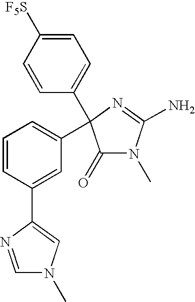

The compound was synthesized as described for example 22 starting from 1-(3-bromophenyl)-2-[4-(pentafluoro-λ⁶-sulfanyl)phenyl]ethane-1,2-dione (120 mg, 0.26 mmol) and T-Methyl-4-(4,4,5,5-tetramethyl-1,3,2-dioxaborolan-2-yl)-1H-pyrazole (64 mg, 0.31 mmol) to give the title compound (31 mg, 26% yield): ¹H NMR (400 MHz, CD₃OD) δ ppm 7.96 (br. s., 1H) 7.88-7.93 (m, 2H) 7.80 (s, 1H) 7.63 (t, J=8.72 Hz, 3H) 7.56-7.59 (m, 1H) 7.44 (t, J=7.83 Hz, 1H) 7.24-7.27 (m, 1H) 3.90 (s, 3H) 3.32 (s, 3H); MS (ES) m/z 472 [M+1]⁺

### Example 45

### 5-(3-{2-amino-1-methyl-5-oxo-4-[4-(pentafluoro-λ⁶-sulfanyl)phenyl]-4,5-dihydro-1H-imidazol-4-yl}phenyl)pyridine-3-carbonitrile

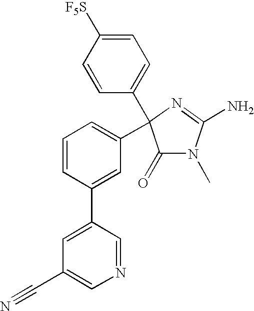

The compound was synthesized as described for example 22 starting from 1-(3-bromophenyl)-2-[4-(pentafluoro-λ⁶-sulfanyl)phenyl]ethane-1,2-dione (120 mg, 0.26 mmol) and 5-cyanopyridin-3-ylboronic acid (45 mg, 0.31 mmol) to give the title compound (76 mg, 60% yield): ¹H NMR (400 MHz, CD₃OD) δ ppm 8.97 (d, J=2.02 Hz, 1 H) 8.82 (d, J=2.02 Hz, 1H) 8.35 (t, J=2.02 Hz, 1H) 7.69-7.77 (m, 3H) 7.58-7.65 (m, 3H) 7.52-7.57 (m, 1H) 7.44-7.51 (m, 1H) 3.13 (s, 3H); MS (ES) m/z 494 [M+1]⁺

### Example 46

### 2-amino-5-(3′-chloro-2′-fluorobiphenyl-3-yl)-3-methyl-5-[4-(pentafluoro-λ⁶-sulfanyl)phenyl]-3,5-dihydro-4H-imidazol-4-one

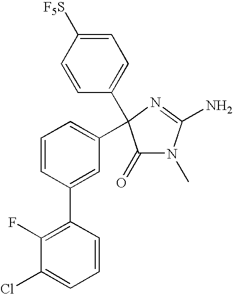

The compound was synthesized as described for example 22 starting from 1-(3-bromophenyl)-2-[4-(pentafluoro-λ⁶-sulfanyl)phenyl]ethane-1,2-dione (120 mg, 0.26 mmol) and 3-Chloro-2-fluorophenylboronic acid (53 mg, 0.31 mmol) to give the title compound (68 mg, 51% yield): ¹H NMR (400 MHz, CD₃OD) δ ppm 7.73-7.82 (m, 2H) 7.54-7.65 (m, 3H) 7.37-7.50 (m, 4H) 7.32 (t, 1H) 7.17 (t, 1H) 3.01 (s, 3H); MS (ES) m/z 520 [M+1]⁺

### Example 47

### 2-amino-5-(2′,6′-difluoro-3′-methoxybiphenyl-3-yl)-3-methyl-5-[4-(pentafluoro-λ⁶-sulfanyl)phenyl]-3,5-dihydro-4H-imidazol-4-one

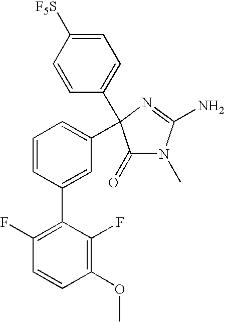

The compound was synthesized as described for example 22 starting from 1-(3-bromophenyl)-2-[4-(pentafluoro-16-sulfanyl)phenyl]ethane-1,2-dione (150 mg, 0.32 mmol) and 2,6-Difluoro-3-methoxyphenylboronic acid (60 mg, 0.32 mmol) to give the title compound (13 mg, 7.6% yield): ¹H NMR (400 MHz, CD₃OD) δ ppm 7.76-7.84 (m, 2H) 7.54-7.65 (m, 2H) 7.33-7.48 (m, 4H) 7.02-7.14 (m, 1H) 6.89-7.00 (m, 1H) 3.84 (s, 3H) 3.10 (s, 3H); MS (ES) m/z 534 [M+1]⁺

### Example 48

### 2-Amino-5-(4-fluoro-3-pyrimidin-5-ylphenyl)-3-methyl-5-[3-(pentafluoro-λ⁶-sulfanyl)phenyl]-3,5-dihydro-4H-imidazol-4-one 0.25 acetate

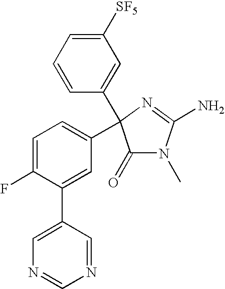

The compound was synthesized as described for Example 22 starting from 2-amino-5-(3-bromo-4-fluorophenyl)-3-methyl-5-[3-(pentafluoro-λ⁶-sulfanyl)phenyl]-3,5-dihydro-4H-imidazol-4-one (200 mg, 0.41 mmol) to give the title compound (64.0 mg, 31% yield): ¹H NMR (400 MHz, CDCl₃) δ ppm 9.21 (s, 1H); 8.90 (s, 2H); 7.98 (s, 1H); 7.74 (d, J=7.8 Hz, 1H); 7.68 (dd, J=8.1, 1.5 Hz, 1H); 7.57-7.63 (m, 2H); 7.44 (t, J=8.0 Hz, 1H); 7.19 (t, J=9.3 Hz, 1H); 5.65 (br. s., 2H); 3.14 (s, 3H); 2.10 (s, 1H); MS (ES) m/z 488,0 [M+H]⁺.

### Example 49

### 5-(5-{2-Amino-1-methyl-5-oxo-4-[3-(pentafluoro-λ⁶-sulfanyl)phenyl]-4,5-dihydro-1H-imidazol-4-yl}-2-fluorophenyl)nicotinonitrile 0.25 acetate

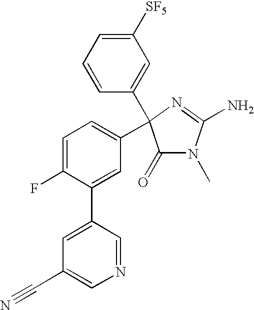

The compound was synthesized as described for Example 22 starting from 2-amino-5-(3-bromo-4-fluorophenyl)-3-methyl-5-[3-(pentafluoro-λ⁶-sulfanyl)phenyl]-3,5-dihydro-4H-imidazol-4-one (150 mg, 0.31 mmol) and 5-(4,4,5,5-tetramethyl-1,3,2-dioxaborolan-2-yl)nicotinonitrile (0.071 g, 0.31 mmol) to give the title compound (64.0 mg, 31% yield): 1 ¹H NMR (400 MHz, CD₃OD) δ ppm 8.88-8.97 (m, 2H); 8.36 (d, J=1.0 Hz, 1H); 7.92 (t, J=1.9 Hz, 1H); 7.68-7.80 (m, 2H); 7.50-7.60 (m, 3H); 7.29 (dd, J=10.2, 8.7 Hz, 1H); 3.13 (s, 3H) 2.03 (s, 0.25H); MS (ES) m/z 512.0 [M+H]⁺.

### Example 50

### 2-Amino-5-(4-fluoro-3-pyridin-3-ylphenyl)-3-methyl-5-[3-(pentafluoro-λ⁶-sulfanyl)phenyl]-3,5-dihydro-4H-imidazol-4-one

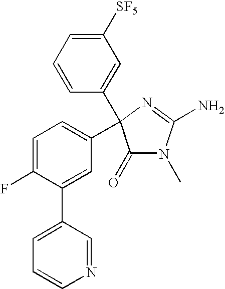

The title compound was synthesized as described for Example 22 starting from 2-amino-5-(3-bromo-4-fluorophenyl)-3-methyl-5-[3-(pentafluoro-λ⁶-sulfanyl)phenyl]-3,5-dihydro-4H-imidazol-4-one (150 mg, 0.31 mmol) and pyridin-3-ylboronic acid (37.8 mg, 0.31 mmol) to give the title compound (72.0 mg, 48% yield): ¹H NMR (400 MHz, CD₃OD) δ ppm 8.66 (s, 1H); 8.54 (dd, J=4.9, 1.4 Hz, 1H); 7.98 (dd, J=8.1, 1.5 Hz, 1H); 7.92 (t, J=1.9 Hz, 1H); 7.70-7.79 (m, 2H); 7.45-7.57 (m, 4H); 7.26 (dd, J=10.1, 8.6 Hz, 1H); 3.14 (s, 3H); MS (ES) m/z 487.1 [M+H]⁺.

### Example 51

### 2-Amino-5-[4-fluoro-3-(5-methoxypyridin-3-yl)phenyl]-[3-methyl-5-3-(pentafluoro-6-sulfanyl)phenyl]-3,5-dihydro-4H-imidazol-4-one

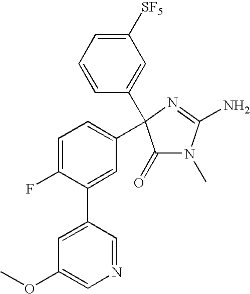

The compound was synthesized as described for Example 22 starting from 2-amino-5-(3-bromo-4-fluorophenyl)-3-methyl-5-[3-(pentafluoro-λ⁶-sulfanyl)phenyl]-3,5-dihydro-4H-imidazol-4-one (150 mg, 0.31 mmol) and 3-methoxy-5-(4,4,5,5-tetramethyl-1,3,2-dioxaborolan-2-yl)pyridine (72.2 mg, 0.31 mmol) to give the title compound (82 mg, 52% yield): ¹H NMR (400 MHz, CD₃OD) δ ppm 8.25-8.28 (m, 2H); 7.94 (s, 1H); 7.72-7.80 (m, 2H); 7.48-7.59 (m, 4H); 7.27 (dd, J=10.2, 8.7 Hz, 1H); 3.94 (s, 3H); 3.16 (s, 3H): MS (ES) m/z 517.1 [M+H]⁺.

### Example 52

### 2-Amino-5-[4-fluoro-3-(5-fluoropyridin-3-yl)phenyl]-3-methyl-5-[3-(pentafluoro-λ⁶-sulfanyl)phenyl]-3,5-dihydro-4H-imidazol-4-one

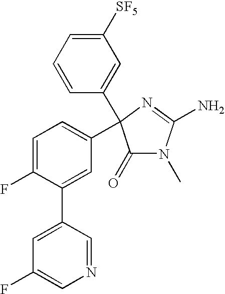

The compound was synthesized as described for Example 22 starting from 2-amino-5-(3-bromo-4-fluorophenyl)-3-methyl-5-[3-(pentafluoro-λ⁶-sulfanyl)phenyl]-3,5-dihydro-4H-imidazol-4-one (150 mg, 0.31 mmol) and 3-fluoro-5-(4,4,5,5-tetramethyl-1,3,2-dioxaborolan-2-yl)pyridine (69 mg, 0.31 mmol) to give the title compound (84 mg, 54% yield): 1H NMR (400 MHz, CD₃OD) δ ppm 8.53 (d, J=10.3 Hz, 1H); 8.48 (d, J=2.5 Hz, 1 H); 7.92 (t, J=1.9 Hz, 1H); 7.69-7.84 (m, 3H); 7.55 (dd, J=7.3, 2.3 Hz, 2H); 7.48-7.53 (m, 1H); 7.27 (dd, J=10.2, 8.7 Hz, 1H); 3.14 (s, 3H); MS (ES) m/z 505.0 [M+H]⁺.

### Assays

Compounds were tested in at least one of the following assays:

### β-Secretase Enzyme

The enzyme used in the IGEN Cleavage-, Fluorescent-, TR-FRET- and BiaCore assays is described as follows:

The soluble part of the human β-Secretase (AA 1-AA 460) was cloned into the ASP2-Fc10-1-IRES-GFP-neoK mammalian expression vector. The gene was fused to the Fc domain of IgG1 (affinity tag) and stably cloned into SEEK 293 cells. Purified sBACE-Fc is stored in Tris buffer, pH 9.2 and has a purity of 95%.

### IGEN Cleavage Assay

The enzyme was diluted to 43 μg/ml in 40 mM MES pH 5.0. The IGEN substrate was diluted to 12 μM in 40 mM MES pH 5.0. Compounds were diluted to the desired concentration in dimethyl sulfoxide (final dimethyl sulfoxide concentration in assay is 5%). The assay was performed in a 96 well PCR plate from Greiner (#650201). Compound in dimethyl sulfoxide (3 μL) and enzyme (27 μL) were added to the plate, and pre-incubated for 10 min. The reaction was started with substrate (30 μL). The final dilution of enzyme was 20 μg/ml and the final concentration of substrate was 6 μM. After 20 minutes reaction at room temperature (RT), the reaction was stopped by removing 10 μL of the reaction mix and diluting it 1:25 in 0.2 M Trizma-HCl, pH 8.0. The product was quantified by adding 50 μL of a 1:5000 dilution of the neoepitope antibody to 50 μL of the 1:25 dilution of the reaction mix (all antibodies and the streptavidin coated beads were diluted in PBS containing 0.5% BSA and 0.5% Tween20). Then, 100 μL of 0.2 mg/mL streptavidin coated beads (Dynabeads M-280) and a 1:5000 dilution of ruthenylated goat anti-rabbit (Ru-GαR) antibody was added. The mixture was measured for electro-chemiluminescence in a BioVeris M8 Analyzer after 2 hours of incubation with shaking at RT. The dimethyl sulfoxide control defined 100% activity level and 0% activity was defined by exclusion of the enzyme (using 40 mM MES pH 5.0 buffer instead).

### Fluorescent Assay

The enzyme was diluted to 52 μg/ml in 40 mM MES pH 5.0. The substrate (Dabcyl-Edans) was diluted to 30 μM in 40 mM MES pH 5.0. Compounds were diluted to the desired concentration in dimethyl sulfoxide (final dimethyl sulfoxide concentration in assay is 5%). The assay is done in a Corning 384 well round bottom, low volume, non-binding surface plate (Corning #3676). Enzyme (9 μL) together with 1 μL of compound in dimethyl sulfoxide were added to the plate and pre-incubated for 10 min. Substrate (10 μL) was added and the reaction proceeded in the dark at RT for 25 min. The final dilution of enzyme was 23 μg/ml, and the final concentration of substrate was 15 μM (Km of 25 μM). The fluorescence of the product was measured on a Victor II plate reader with an excitation wavelength of 360 nm and an emission wavelength of 485 nm using a protocol for labelled Edans peptide. The dimethyl sulfoxide control defined 100% activity level and 0% activity was defined by exclusion of the enzyme (using 40 mM MES pH 5.0 buffer instead).

### TR-FRET Assay

Enzyme was diluted to 6 μg/mL and the substrate (Europium)CEVNLDAEFK(Qsy7) to 200 nM in reaction buffer (NaAcetate, chaps, triton x-100, EDTA pH 4.5). Compounds were diluted to the desired concentration in dimethyl sulfoxide (final dimethyl sulfoxide concentration in assay is 5%). The assay was done in a Costar 384 well round bottom, low volume, non-binding surface plate (Corning #3676). Enzyme (9 μL) and 1 μL of compound in dimethyl sulfoxide was added to the plate, mixed and pre-incubated for 10 min. Substrate (10 μL) was added and the reaction proceeded in the dark for 15 min at RT. The reaction was stopped with the addition of 7 μL NaAcetate, pH 9. The fluorescence of the product was measured on a Victor II plate reader with an excitation wavelength of 340 nm and an emission wavelength of 615 nm. The final concentration of the enzyme was 2.7 μg/ml and the final concentration of the substrate was 100 nM (Kin of 290 nM). The dimethyl sulfoxide control defined the 100% activity level and 0% activity was defined by exclusion of the enzyme (using reaction buffer instead).

### BACE Biacore Sensor Chip Preparation

BACE was assayed on a Biacore3000 instrument by attaching either a peptidic transition state isostere (TSI) or a scrambled version of the peptidic TSI to the surface of a Biacore CM5 sensor chip. The surface of a CM5 sensor chip has 4 distinct channels that can be used to couple the peptides. The scrambled peptide KFES-statinc-ETIAEVENV was coupled to channel 1 and the TSI inhibitor KTEEISEVN-statine-VAEF was coupled to channel 2 of the same chip. The two peptides were dissolved at 0.2 mg/mL in 20 mM sodium acetate pH 4.5, and then the solutions were centrifuged at 14K rpm to remove any particulates. Carboxyl groups on the dextran layer were activated by injecting a one to one mixture of 0.5 M N-ethyl-N′ (3-dimethylaminopropyl)-carbodiimide and 0.5 M N-hydroxysuccinimide at 5 μL/min for 7 min. Then the stock solution of the control peptide was injected in channel 1 for 7 min at 5 μL/min., and then the remaining activated carboxyl groups were blocked by injecting 1 M ethanolamine for 7 min at 5 μL/min.

### BACE Biacore Assay Protocol

The BACE Biacore assay was done by diluting BACE to 0.5 μM in sodium acetate buffer at pH 4.5 (running buffer minus dimethyl sulfoxide). The diluted BACE was mixed with dimethyl sulfoxide or compound diluted in dimethyl sulfoxide at a final concentration of 5% dimethyl sulfoxide. The BACE/inhibitor mixture was incubated for 30 minutes at RT before being injected over channel 1 and 2 of the CM5 Biacore chip at a rate of 20 μL/min. As BACE bound to the chip the signal was measured in response units (RU). BACE binding to the TSI inhibitor on channel 2 gave a certain signal. The presence of a BACE inhibitor reduced the signal by binding to BACE and inhibiting the interaction with the peptidic TSI on the chip. Any binding to channel 1 was non-specific and was subtracted from the channel 2 responses. The dimethyl sulfoxide control was defined as 100% and the effect of the compound was reported as percent inhibition of the dimethyl sulfoxide control.

### Beta-Secretase Whole Cell Assays

### Generation of HEK293-APP695

The pcDNA3.1 plasmid encoding the cDNA of human full-length APP695 was stably transfected into HEK-293 cells using the Lipofectamine transfection reagent according to manufacture's protocol (Invitrogen). Colonies were selected with 0.1-0.5 mg/ml, of zeocin. Limited dilution cloning was performed to generate homogeneous cell lines. Clones were characterized by levels of APP expression and Aβ secreted in the conditioned media using an ELISA assay developed in-house.

### Cell Culture for HEK293-APP695

HEK293 cells stably expressing human wild-type APP (HEK293-APP695) were grown at 37° C., 5% CO— in DMEM containing 4500 g/l, glucose, GlutaMAX and sodium pyruvate supplemented with 10% FBS, 1% non-essential amino acids and 0.1 mg/mL of the selection antibiotic zeocin.

### Aβ40 Release Assay

HEK293-APP695 cells were harvested at 80-90% confluence and seeded at a concentration of 0.2×10⁶ cells/mL, 100 mL cell suspension/well, onto a black clear bottom 96-well poly-D-lysine coated plate. After over night incubation at 37° C., 5% CO₂, the cell medium was replaced with cell culture medium with penicillin and streptomycin (100 U/mL, 100 μg/mL, respectively) containing test compounds in a final dimethyl sulfoxide concentration of 1%. Cells were exposed to the test compounds for 24 h at 37° C., 5% CO₂. To quantify the amount of released Aβ, 100 μL cell medium was transferred to a round bottom polypropylene 96-well plate (assay plate). The cell plate was saved for the ATP assay, as described below. To the assay plate, 50 μL of primary detection solution containing 0.5 μg/mL of the rabbit anti-Aβ40 antibody and 0.5 μg/mL of the biotinylated monoclonal mouse 6E10 antibody in DPBS with 0.5% BSA and 0.5% Tween-20 was added per well and incubated over night at 4° C. Then, 50 μL of secondary detection solution containing 0.5 μg/mL of a ruthenylated goat anti-rabbit antibody and 0.2 mg/mL of streptavidin coated beads (Dynabeads M-280) was added per well. The plate was vigorously shaken at RT for 1-2 hours. The plate was then measured for electro-chemiluminescence in a BioVeris M8 Analyzer.

### Cell Culture for SH-SY5Y

SH-SY5Y cells were grown 37° C. with 5% CO₂ in DMEM/F-12 1:1 containing GlutaMAX supplemented with 1 mM HEPES, 10% FBS and 1% non-essential amino acids.

sAPPβ Release Assay

SH-SY5Y cells were harvested at 80-90% confluence and seeded at a concentration of 1.5×10⁶ cells/mL, 100 mL cell suspension/well, onto a black clear flat bottom 96-well tissue culture plate. After 7 hours of incubation at 37° C., 5% CO₂, the cell medium was replaced with 90 μl cell culture medium with penicillin and streptomycin (100 U/mL, 100 μg/mL, respectively) containing test compounds in a final dimethyl sulfoxide concentration of 1%. Cells were exposed to the test compounds for 18 h at 37° C., 5% CO₂. To measure sAPPβ released into the cell medium, sAPPβ microplates from Meso Scale Discovery (MSD) were used and the assay was performed according to the manufacture's protocol. Briefly, 25 μL cell medium was transferred to a previously blocked MSD sAPPβ microplate. The cell plate was saved for the ATP assay, as described below. The sAPPβ was captured during shaking at RT for 1 hour, by antibodies spotted in the wells of the microplate. After multiple washes, SULFO-TAG labeled detection antibody was added (25 μL/well, final concentration 1 nM) to the assay plate and the plate was incubated with shaking at RT for 1 hour. Following multiple washes, 150 μl/well of Read Buffer T was added to the plate. After 10 minutes at RT the plate was read in the SECTOR™ Imager for electro-chemiluminescence.

### ATP Assay

As indicated above, after transferring medium for analysis of Aβ40 or sAPPβ from the cell plate, the plate was used to analyze cytotoxicity using the ViaLight™ Plus cell proliferation/cytotoxicity kit from Cambrex BioScience that measures total cellular ATP. The assay was performed according to the manufacture's protocol. Briefly, 50 μL cell lysis reagent was added per well. The plates were incubated at RT for 10 min. Two min after addition of 100 μL reconstituted ViaLight™ Plus ATP reagent, the luminescence was measured in a Wallac Victor² 1420 multilabel counter.

hERG Assay

### Cell Culture

The hERG-expressing Chinese hamster ovary K1 (CHO) cells described by (Persson, Carlsson, Duker, & Jacobson, 2005) were grown to semi-confluence at 37° C. in a humidified environment (5% CO₂) in F-12 Ham medium containing L-glutamine, 10% foetal calf serum (FCS) and 0.6 mg/ml hygromycin (all Sigma-Aldrich). Prior to use, the monolayer was washed using a pre-warmed (37° C.) 3 ml aliquot of Versene 1:5,000 (Invitrogen). After aspiration of this solution the flask was incubated at 37° C. in an incubator with a further 2 ml of Versene 1:5,000 for a period of 6 minutes. Cells were then detached from the bottom of the flask by gentle tapping and 10 ml of Dulbecco's Phosphate-Buffered Saline containing calcium (0.9 mM) and magnesium (0.5 mM) (PBS; Invitrogen) was then added to the flask and aspirated into a 15 ml centrifuge tube prior to centrifugation (50 g, for 4 mins). The resulting supernatant was discarded and the pellet gently re-suspended in 3 ml of PBS. A 0.5 ml aliquot of cell suspension was removed and the number of viable cells (based on trypan blue exclusion) was determined in an automated reader (Cedex; Innovatis) so that the cell re-suspension volume could be adjusted with PBS to give the desired final cell concentration. It is the cell concentration at this point in the assay that is quoted when referring to this parameter. CHO-Kv1.5 cells, which were used to adjust the voltage offset on IonWorks™ HT, were maintained and prepared for use in the same way.

### Electrophysiology

The principles and operation of this device have been described by (Schroeder, Neagle, Trezise, & Worley, 2003). Briefly, the technology is based on a 384-well plate (PatchPlate™) in which a recording is attempted in each well by using suction to position and hold a cell on a small hole separating two isolated fluid chambers. Once sealing has taken place, the solution on the underside of the PatchPlate™ is changed to one containing amphotericin B. This permeablises the patch of cell membrane covering the hole in each well and, in effect, allows a perforated, whole-cell patch clamp recording to be made.

A β-test IonWorks™ HT from Essen Instrument was used. There is no capability to warm solutions in this device hence it was operated at room temperature (˜21° C.), as follows. The reservoir in the “Buffer” position was loaded with 4 ml of PBS and that in the “Cells” position with the CHO-hERG cell suspension described above. A 96-well plate (V-bottom, Greiner Bio-one) containing the compounds to be tested (at 3-fold above their final test concentration) was placed in the “Plate 1” position and a PatchPlate™ was clamped into the PatchPlate™ station. Each compound plate was laid-out in 12 columns to enable ten, 8-point concentration-effect curves to be constructed; the remaining two columns on the plate were taken up with vehicle (final concentration 0.33% DMSO), to define the assay baseline, and a supra-maximal blocking concentration of cisapride (final concentration 10 μM) to define the 100% inhibition level. The fluidics-head (F-Head) of IonWorks™ HT then added 3.5 μl of PBS to each well of the PatchPlate™ and its underside was perfused with “internal” solution that had the following composition (in mM): K-Gluconate 100, KCl 40, MgCl₂ 3.2, EGTA 3 and HEPES 5 (all Sigma-Aldrich; pH 7.25-7.30 using 10 M KOH). After priming and de-bubbling, the electronics-head (E-head) then moved round the PatchPlate™ performing a hole test (i.e. applying a voltage pulse to determine whether the hole in each well was open). The F-head then dispensed 3.5 μl of the cell suspension described above into each well of the PatchPlate™ and the cells were given 200 seconds to reach and seal to the hole in each well. Following this, the E-head moved round the PatchPlate™ to determine the seal resistance obtained in each well. Next, the solution on the underside of the PatchPlate™ was changed to “access” solution that had the following composition (in mM): KCl 140, EGTA 1, MgCl₂ 1 and HEPES 20 (pH 7.25-7.30 using 10 M KOH) plus 100 μg/ml of amphotericin B (Sigma-Aldrich). After allowing 9 minutes for patch perforation to take place, the E-head moved round the PatchPlate™ 48 wells at a time to obtain pre-compound hERG current measurements. The F-head then added 3.5 μl of solution from each well of the compound plate to 4 wells on the PatchPlate™ (the final DMSO concentration was 0.33% in every well). This was achieved by moving from the most dilute to the most concentrated well of the compound plate to minimise the impact of any compound carry-over. After approximately 3.5 mins incubation, the E-head then moved around all 384-wells of the PatchPlate™ to obtain post-compound hERG current measurements. In this way, non-cumulative concentration-effect curves could be produced where, providing the acceptance criteria were achieved in a sufficient percentage of wells (see below), the effect of each concentration of test compound was based on recording from between 1 and 4 cells.

The pre- and post-compound hERG current was evoked by a single voltage pulse consisting of a 20 s period holding at −70 mV, a 160 ms step to −60 mV (to obtain an estimate of leak), a 100 ms step back to −70 mV, a 1 s step to +40 mV, a 2 s step to −30 mV and finally a 500 ms step to −70 mV. In between the pre- and post-compound voltage pulses there was no clamping of the membrane potential. Currents were leak-subtracted based on the estimate of current evoked during the +10 mV step at the start of the voltage pulse protocol. Any voltage offsets in IonWorks™ HT were adjusted in one of two ways. When determining compound potency, a depolarising voltage ramp was applied to CHO-Kv1.5 cells and the voltage noted at which there was an inflection point in the current trace (i.e. the point at which channel activation was seen with a ramp protocol). The voltage at which this occurred had previously been determined using the same voltage command in conventional electrophysiology and found to be −15 mV (data not shown); thus an offset potential could be entered into the IonWorks™ HT software using this value as a reference point. When determining the basic electrophysiological properties of hERG, any offset was adjusted by determining the hERG tail current reversal potential in IonWorks™ HT, comparing it with that found in conventional electrophysiology (−82 mV) and then making the necessary offset adjustment in the IonWorks™ HT software. The current signal was sampled at 2.5 kHz.

Pre- and post-scan hERG current magnitude was measured automatically from the leak subtracted traces by the IonWorks™ HT software by taking a 40 ms average of the current during the initial holding period at −70 mV (baseline current) and subtracting this from the peak of the tail current response. The acceptance criteria for the currents evoked in each well were: pre-scan seal resistance >60 MΩ, pre-scan hERG tail current amplitude >150 pA; post-scan seal resistance >60 MΩ. The degree of inhibition of the hERG current was assessed by dividing the post-scan hERG current by the respective pre-scan hERG current for each well.

### Results

Typical Kᵢ values for the compounds of the present invention are in the range of about 1 to about 2,000 nM. Biological data on final compounds are given below in Table 1.

TABLE 1

| | IC50 (nM) in |
|---|---|
| Example No. | TR-FRET assay |
| 5 | 756 |
| 22 | 49 |
| 23 | 101 |
| 24 | 53 |
| 25 | 30 |
| 26 isomer 1 | 13 |
| 26 isomer 2 | 465 |
| 27 | 81 |
| 28 | 69 |
| 29 | 81 |
| 30 | 124 |
| 31 | 83 |
| 32 | 55 |
| 33 | 52 |
| 34 | 467 |
| 35 | 68 |
| 36 | 56 |
| 37 isomer 1 | 49 |
| 37 isomer 2 | 2630 |
| 38 | 71 |
| 39 | 356 |
| 40 | 81 |
| 41 | 143 |
| 42 | 405 |
| 43 | 673 |
| 44 | 643 |
| 45 | 59 |
| 46 | 694 |
| 47 | 38 |
| 48 | 487 |
| 49 | 1140 |
| 50 | 317 |
| 51 | 305 |
| 52 | 1190 |

1. A compound according to Formula I 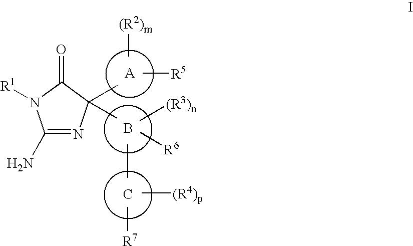
wherein
A is independently selected from hydrogen, C₁₋₆alkyl, C₃₋₆alkenyl, C₃₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl and C₀₋₆alkylheterocyclyl, wherein said C₁₋₆alkyl, C₃₋₆alkenyl, C₃₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl or C₀₋₆alkylheterocyclyl is optionally substituted with one or more R⁵;
B is independently selected from aryl and heteroaryl, said aryl or heteroaryl optionally being substituted with one or more R⁶;
C is independently selected from hydrogen, cycloalkyl, cycloalkenyl, cycloalkynyl, aryl, heteroaryl and heterocyclyl, wherein said cycloalkyl, cycloalkenyl, cycloalkynyl, aryl, heteroaryl or heterocyclyl is optionally substituted with one or more R⁷;
R¹ is selected from hydrogen, C₁₋₆alkyl, C₃₋₆alkenyl, C₃₋₆alkynyl, C₃₋₆cycloalkyl, cycloalkenyl, cycloalkynyl, aryl, heteroaryl, heterocyclyl and C₁₋₆alkylcycloalkyl, wherein said C₁₋₆alkyl, C₃₋₆alkenyl, C₃₋₆alkynyl, C₃₋₆cycloalkyl, C₅₋₇cycloalkenyl, C₅₋₇cycloalkynyl, aryl, heteroaryl or heterocyclyl, is optionally substituted with one or more D;
R², R³ and R⁴ is independently selected from N═(SO)R⁸R⁹, SF₅, and OSF₅;
R⁵, R⁶ and R⁷ is independently selected from hydrogen, halogen, nitro, CHO, C₀₋₆alkylCN, OC₁₋₆alkylCN, C₀₋₆alkylOR¹⁰, OC₂₋₆alkylOR¹⁰, C₀₋₆alkylNR¹⁰R¹¹, OC₂₋₆alkylNR¹⁰R¹¹, OC₂₋₆alkylOC₂₋₆alkylNR¹⁰R¹¹, NR¹⁰R¹¹, C₀₋₆alkylCO₂R¹⁰, OC₁₋₆alkylCO₂R¹⁰, C₀₋₆alkylCONR¹⁰R¹¹, OC₁₋₆alkylCONR¹⁰R¹¹, OC₂₋₆alkylNR¹⁰(CO)R¹¹, C₀₋₆alkylNR¹⁰(CO)R¹¹, O(CO)NR¹⁰R¹¹, NR¹⁰(CO)OR¹¹, NR¹⁰(CO)NR¹⁰R¹¹, O(CO)OR¹¹, O(CO)R¹⁰, C₀₋₆alkylCOR¹⁰, OC₁₋₆alkylCOR¹⁰, NR¹⁰(CO)(CO)R¹⁰, NR¹⁰(CO)(CO)NR¹⁰R¹¹, C₀₋₆alkylSR¹⁰, C₀₋₆alkyl(SO₂)NR¹⁰R¹¹, OC₁₋₆alkylNR¹⁰(SO₂)R¹¹, OC₀₋₆alkyl(SO₂)NR¹⁰R¹¹, C₀₋₆alkyl(SO)NR¹⁰R¹¹, OC₁₋₆alkyl(SO)NR¹⁰R¹¹, OSO₂R¹⁰, SO₃R¹⁰, C₀₋₆alkylNR¹⁰(SO₂)NR¹⁰R¹¹, C₀₋₆alkylNR¹⁰(SO)R¹¹, OC₂₋₆alkylNR¹⁰(SO)R¹¹, OC₁₋₆alkylSO₂R¹⁰, C₁₋₆alkylSO₂R¹⁰, C₀₋₆alkylSOR¹⁰, C₁₋₆alkyl, C₂₋₆alkenyl, C₂₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl and C₀₋₆alkylheterocyclyl, wherein said C₁₋₆alkyl, C₂₋₆alkenyl, C₂₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl or C₀₋₆alkylheterocyclyl is optionally substituted by one or more D;
R⁸ and R⁹ is independently selected from hydrogen, C₁₋₆alkyl, C₂₋₆alkenyl, C₂₋₆alkynyl, cycloalkyl, cycloalkenyl, cycloalkynyl, aryl, heteroaryl and heterocyclyl, wherein said C₁₋₆alkyl, C₂₋₆alkenyl, C₂₋₆alkynyl, cycloalkyl, cycloalkenyl, cycloalkynyl, aryl, heteroaryl or heterocyclyl is optionally substituted by one or more D; or
R⁸ and R⁹ may together form a 3 to 7 membered heterocyclic ring containing one or more heteroatoms selected from N, O or S, wherein said heterocyclic ring is optionally substituted by one or more D;
R¹⁰ and R¹¹ is independently selected from hydrogen, halogen, C₁₋₆alkyl, C₂₋₆alkenyl, C₂₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl, C₀₋₆alkylheterocyclyl, C₀₋₆alkylOR¹² and C₀₋₆alkylNR¹²R¹³, wherein said C₁₋₆alkyl, C₂₋₆alkenyl, C₂₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl or C₀₋₆alkylheterocyclyl is optionally substituted by one or more D; or
R¹⁰ and R¹¹ may together form a 4 to 6 membered heterocyclic ring containing one or more heteroatoms selected from N, O or S, wherein said heterocyclic ring is optionally substituted by one or more D;
R¹² and R¹³ is independently selected from hydrogen, C₁₋₆alkyl, C₃₋₆alkenyl, C₃₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheterocyclyl and C₀₋₆alkylheteroaryl, wherein said C₁₋₆alkyl, C₃₋₆alkenyl, C₃₋₆alkynyl, C₀₋₆alkylcycloalkyl, C₀₋₆alkylcycloalkenyl, C₀₋₆alkylcycloalkynyl, C₀₋₆alkylaryl, C₀₋₆alkylheteroaryl or C₀₋₆alkylheterocyclyl is optionally substituted by one or more D; or
R¹² and R¹³ may together form a 4 to 6 membered heterocyclic ring containing one or more heteroatoms selected from N, O or S wherein said heterocyclic ring is optionally substituted by one or more D;
D is independently selected from halogen, nitro, CN, OR¹⁴, C₁₋₆alkyl, C₂₋₆alkenyl, C₂₋₆alkynyl, C₀₋₆alkylaryl, C₀₋₆alkylheteroaryl, C₀₋₆alkylcycloalkyl, C₀₋₆alkylcycloalkenyl, C₀₋₆alkylcycloalkynyl, C₀₋₆alkylheterocyclyl, OC₂₋₆alkylNR¹⁴R¹⁵, NR¹⁴R¹⁵, CONR¹⁴R¹⁵, NR¹⁴(CO)R¹⁵, O(CO)C₁₋₆alkyl, (CO)OC₁₋₆alkyl, COR¹⁴, (SO₂)NR¹⁴R¹⁵, NSO₂R¹⁴, SO₂R¹⁴, SOR¹⁴, (CO)C₁₋₆alkylNR¹⁴R¹⁵, (SO₂)C₁₋₆alkylNR¹⁴R¹⁵, OSO₂R¹⁴ and SO₃R¹⁵, wherein said C₁₋₆alkyl, C₂₋₆alkenyl, C₂₋₆alkynyl, C₀₋₆alkylaryl, C₀₋₆alkylheteroaryl, C₀₋₆alkylheterocyclyl, C₀₋₆alkylcycloalkyl C₀₋₆alkylcycloalkenyl or C₀₋₆alkylcycloalkynyl is optionally substituted with halogen, nitro, CN, C₁₋₆alkyl, OR¹⁴, OSO₂R¹⁴ or SO₃R¹⁴;
R¹⁴ and R¹⁵ is independently selected from hydrogen, halogen, C₁₋₆alkyl, C₂₋₆alkenyl, C₂₋₆alkynyl, C₃₋₆cycloalkyl, aryl, heteroaryl and heterocyclyl; or
R¹⁴ and R¹⁵ may together form a 4 to 6 membered heterocyclic ring containing one or more heteroatoms selected from N, O or S;
m=0, 1, 2 or 3;
n=0, 1, 2 or 3;
p=0, 1, 2 or 3;
wherein one of m, n or p is at least 1;
as a free base or a pharmaceutically acceptable salt, solvate or solvate of a salt thereof.

2. A compound according to claim 1, wherein R¹ is C₁₋₆alkyl.

3. A compound according to claim 1, wherein R¹ is methyl.

4. A compound according to claim 1, wherein A is C₀₋₆alkylaryl, said C₀₋₆alkylaryl being optionally substituted with one or more R⁵.

5. A compound according to claim 1, wherein A represents phenyl.

6. A compound according to claim 1, wherein R⁵ is selected from hydrogen and C₀₋₆alkylOR¹⁰.

7. A compound according to claim 1, wherein said C₀₋₆alkylOR¹⁰ represents methoxy.

8. A compound according to claim 1, wherein B is aryl, optionally substituted with one R⁶.

9. A compound according to claim 1, wherein said B represents phenyl substituted with one fluoro.

10. A compound according to claim 1, wherein C is selected from aryl and heteroaryl, wherein said aryl or heteroaryl is optionally substituted with one or more R⁷.

11. A compound according to claim 1, wherein R⁷ is selected from hydrogen, halogen, C₀₋₆alkylCN and C₀₋₆alkylOR¹⁰.

12. A compound according to claim 1, wherein C represents pyrimidyl.

13. A compound according to claim 1, wherein C represents phenyl substituted with one methoxy.

14. A compound according to claim 1, wherein C represents pyridyl.

15. A compound according to claim 1, wherein C represents pyridyl substituted with one methoxy, one cyano or one fluoro.

16. A compound according to claim 1, wherein
m=0 or 1;
n=0;
p=0 or 1;
wherein one of m or p is least 1.

17. A compound according to claim 1, wherein m is 1 and R² is independently selected from N═(SO)R⁸R⁹ and SF₅.

18. A compound according to claim 1, wherein R⁸ and R⁹ represents methyl.

19. A compound according to claim 1, wherein p is 1 and R⁴ is N═(SO)R⁸R⁹.

20. A compound according to claim 1, wherein m is 1 and R² is SF₅.

21. A compound according to claim 1, wherein
A is C₀₋₆alkylaryl, optionally substituted with one R⁵;
B is aryl, optionally substituted with one or more R⁶;
C is aryl or heteroaryl, wherein said aryl or heteroaryl is optionally substituted with one R⁷;
R¹ is C₁₋₆alkyl;
R², R³ and R⁴ is independently selected from N═(SO)R⁸R⁹ and SF₅;
R⁵, R⁶ and R⁷ is independently selected from hydrogen, halogen and C₀₋₆alkylOR¹⁰; C₀₋₆alkylCN;
R⁸ and R⁹ is C₁₋₆alkyl;
R¹⁰ is C₁₋₆alkyl;
m=0 or 1;
n=0;
p=0 or 1;
wherein one of m or p is 1.

22. A compound according to claim 1, wherein
A is phenyl;
B is phenyl, optionally substituted with one or more R⁶;
C is aryl or heteroaryl, wherein said aryl or heteroaryl is optionally substituted with one
R⁷;
R¹ is C₁₋₆alkyl;
R² is SF₅;
R⁶ and R⁷ is independently selected from hydrogen, halogen, C₁₋₆alkyl, C₀₋₆alkylOR¹⁰;
C₀₋₆alkylCN;
m=1;
n=0;
p=0; and
R¹⁰ represents methyl.

23. A compound according to claim 22, wherein C is a heteroaryl selected from pyridine, pyrimidine, pyrazine, thiazole and pyrazole.

24. A compound according to claim 22, wherein C is a phenyl, substituted with one, two or three R⁷, independently selected from halogen, cyano and methoxy.

25. A compound according to claim 1, selected from:
2-Amino-5-(4-{[dimethyl(oxido)-λ⁴-sulfanylidene]amino}phenyl)-5-(6-fluoro-3′-methoxybiphenyl-3-yl)-3-methyl-3,5-dihydro-4H-imidazol-4-one hydrochloride;
2-Amino-5-(3′-{[dimethyl(oxido)-λ⁴-sulfanylidene]amino}-5′-methoxybiphenyl-3-yl)-3-methyl-5-phenyl-3,5-dihydro-4H-imidazol-4-one hydrochloride;
2-Amino-5-(3′-{[dimethyl(oxido)-λ⁴-sulfanylidene]amino}-5′-methoxybiphenyl-3-yl)-5-(4-methoxyphenyl)-3-methyl-3,5-dihydro-4H-imidazol-4-one hydrochloride;
2-Amino-5-(4-fluoro-3-pyrimidin-5-ylphenyl)-3-methyl-5-[4-(pentafluoro-λ⁶-sulfanyl)phenyl]-3,5-dihydro-4H-imidazol-4-one;
5-(5-{2-Amino-1-methyl-5-oxo-4-[4-(pentafluoro-λ⁶-sulfanyl)phenyl]-4,5-dihydro-1H-imidazol-4-yl}-2-fluorophenyl)nicotinonitrile 0.25 acetate;
2-Amino-5-(4-fluoro-3-pyridin-3-ylphenyl)-3-methyl-5-[4-(pentafluoro-λ⁶-sulfanyl)phenyl]-3,5-dihydro-4H-imidazol-4-one;
2-Amino-5-[4-fluoro-3-(5-methoxypyridin-3-yl)phenyl]-3-methyl-5-[4-(pentafluoro-λ⁶-sulfanyl)phenyl]-3,5-dihydro-4H-imidazol-4-one;
2-Amino-5-[4-fluoro-3-(5-methoxypyridin-3-yl)phenyl]-3-methyl-5-[4-(pentafluoro-λ⁶-sulfanyl)phenyl]-3,5-dihydro-4H-imidazol-4-one (isomer 1);
2-Amino-5-[4-fluoro-3-(5-methoxypyridin-3-yl)phenyl]-3-methyl-5-[4-(pentafluoro-λ⁶-sulfanyl)phenyl]-3,5-dihydro-4H-imidazol-4-one (isomer 2);
2-Amino-5-[4-fluoro-3-(5-fluoropyridin-3-yl)phenyl]-3-methyl-5-[4-(pentafluoro-λ⁶-sulfanyl)phenyl]-3,5-dihydro-4H-imidazol-4-one 0.25 acetate;
2-amino-5-[4-fluoro-3-(2-fluoropyridin-3-yl)phenyl]-3-methyl-5-[4-(pentafluoro-λ⁶-sulfanyl)phenyl]-3,5-dihydro-4H-imidazol-4-one 0.25 acetate;
3-(5-{2-amino-1-methyl-5-oxo-4-[4-(pentafluoro-λ⁶-sulfanyl)phenyl]-4,5-dihydro-1H-imidazol-4-yl}-2-fluorophenyl)isonicotinonitrile 0.25 acetate;
2-amino-5-(4-fluoro-3-pyrazin-2-ylphenyl)-3-methyl-5-[4-(pentafluoro-λ⁶-sulfanyl)phenyl]-3,5-dihydro-4H-imidazol-4-one 0.75 acetate;
2-amino-5-[3-(2-fluoropyridin-3-yl)phenyl]-3-methyl-5-[4-(pentafluoro-λ⁶-sulfanyl)phenyl]-3,5-dihydro-4H-imidazol-4-one;
2-amino-3-methyl-5-[4-(pentafluoro-λ⁶-sulfanyl)phenyl]-5-(3-pyrimidin-5-ylphenyl)-3,5-dihydro-4H-imidazol-4-one;
2-amino-3-methyl-5-[4-(pentafluoro-λ⁶-sulfanyl)phenyl]-5-(3-pyridin-3-ylphenyl)-3,5-dihydro-4H-imidazol-4-one;
3-(3-{2-amino-1-methyl-5-oxo-4-[4-(pentafluoro-λ⁶-sulfanyl)phenyl]-4,5-dihydro-1H-imidazol-4-yl}phenyl)pyridine-4-carbonitrile;
2-amino-5-[3-(5-fluoropyridin-3-yl)phenyl]-3-methyl-5-[4-(pentafluoro-λ⁶-sulfanyl)phenyl]-3,5-dihydro-4H-imidazol-4-one;
2-amino-3-methyl-5-[4-(pentafluoro-λ⁶-sulfanyl)phenyl]-5-(3-pyrazin-2-ylphenyl)-3,5-dihydro-4H-imidazol-4-one;
2-amino-5-(2′-fluoro-3′-methoxybiphenyl-3-yl)-3-methyl-5-[4-(pentafluoro-λ⁶-sulfanyl)phenyl]-3,5-dihydro-4H-imidazol-4-one;
2-amino-5-(2′-fluoro-3′-methoxybiphenyl-3-yl)-3-methyl-5-[4-(pentafluoro-λ⁶-sulfanyl)phenyl]-3,5-dihydro-4H-imidazol-4-one (isomer 1);
2-amino-5-(2′-fluoro-3′-methoxybiphenyl-3-yl)-3-methyl-5-[4-(pentafluoro-λ⁶-sulfanyl)phenyl]-3,5-dihydro-4H-imidazol-4-one (isomer 2);
2-amino-5-(2′-fluoro-5′-methoxybiphenyl-3-yl)-3-methyl-5-[4-(pentafluoro-λ⁶-sulfanyl)phenyl]-3,5-dihydro-4H-imidazol-4-one;
2-amino-5-(2′-fluoro-5′-carbonitrile biphenyl-3-yl)-3-methyl-5-[4-(pentafluoro-λ⁶-sulfanyl)phenyl]-3,5-dihydro-4H-imidazol-4-one;
2-amino-5-(3′-methoxybiphenyl-3-yl)-3-methyl-5-[4-(pentafluoro-λ⁶-sulfanyl)phenyl]-3,5-dihydro-4H-imidazol-4-one;
3′-{2-amino-1-methyl-5-oxo-4-[4-(pentafluoro-λ⁶-sulfanyl)phenyl]-4,5-dihydro-1H-imidazol-4-yl}biphenyl-3-carbonitrile;
2-(3-{2-amino-1-methyl-5-oxo-4-[4-(pentafluoro-λ⁶-sulfanyl)phenyl]-4,5-dihydro-1H-imidazol-4-yl}phenyl)pyridine-4-carbonitrile;
2-amino-3-methyl-5-[4-(pentafluoro-λ⁶-sulfanyl)phenyl]-5-[3-(1,3-thiazol-4-yl)phenyl]-3,5-dihydro-4H-imidazol-4-one;
2-amino-3-methyl-5-[3-(1-methyl-1H-imidazol-4-yl)phenyl]-5-[4-(pentafluoro-λ⁶-sulfanyl)phenyl]-3,5-dihydro-4H-imidazol-4-one;
5-(3-{2-amino-1-methyl-5-oxo-4-[4-(pentafluoro-λ⁶-sulfanyl)phenyl]-4,5-dihydro-1H-imidazol-4-yl}phenyl)pyridine-3-carbonitrile;
2-amino-5-(3′-chloro-2′-fluorobiphenyl-3-yl)-3-methyl-5-[4-(pentafluoro-λ⁶-sulfanyl)phenyl]-3,5-dihydro-4H-imidazol-4-one;
2-amino-5-(2′,6′-difluoro-3′-methoxybiphenyl-3-yl)-3-methyl-5-[4-(pentafluoro-λ⁶-sulfanyl)phenyl]-3,5-dihydro-4H-imidazol-4-one;
2-Amino-5-(4-fluoro-3-pyrimidin-5-ylphenyl)-3-methyl-5-[3-(pentafluoro-λ⁶-sulfanyl)phenyl]-3,5-dihydro-4H-imidazol-4-one 0.25 acetate;
5-(5-{2-Amino-1-methyl-5-oxo-4-[3-(pentafluoro-λ⁶-sulfanyl)phenyl]-4,5-dihydro-1H-imidazol-4-yl}-2-fluorophenyl)nicotinonitrile 0.25 acetate;
2-Amino-5-(4-fluoro-3-pyridin-3-ylphenyl)-3-methyl-5-[3-(pentafluoro-λ⁶-sulfanyl)phenyl]-3,5-dihydro-4H-imidazol-4-one;
2-Amino-5-[4-fluoro-3-(5-methoxypyridin-3-yl)phenyl]-3-methyl-5-[3-(pentafluoro-λ⁶-sulfanyl)phenyl]-3,5-dihydro-4H-imidazol-4-one; and
2-Amino-5-[4-fluoro-3-(5-fluoropyridin-3-yl)phenyl]-3-methyl-5-[3-(pentafluoro-λ⁶-sulfanyl)phenyl]-3,5-dihydro-4H-imidazol-4-one as a free base or a pharmaceutically acceptable salt, solvate or solvate of a salt thereof.

26. A pharmaceutical formulation comprising as active ingredient a therapeutically effective amount of a compound according to claim 1 in association with a pharmaceutically acceptable excipient, carrier or diluent.

27-31. (canceled)

32. A method of inhibiting activity of BACE comprising contacting said BACE with a compound according to claim 1.

33. A method of treating or preventing an Aβ-related pathology in a mammal, comprising administering to said patient a therapeutically effective amount of a compound according to claim 1.

34. The method of claim 33, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

35. The method of claim 33, wherein said mammal is a human.

36. A method of treating or preventing an Aβ-related pathology in a mammal, comprising administering to said patient a therapeutically effective amount of a compound according to claim 1 and at least one cognitive enhancing agent, memory enhancing agent, or choline esterase inhibitor.

37. The method of claim 36, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

38. The method of claim 36, wherein said mammal is a human.
